(19) United States
(12) Patent Application Publication
(10) Pub. No.: US 2007/0172472 A1
(43) Pub. Date: Jul. 26, 2007

(54) Methods and Systems for Treating Injured Cardiac Tissue

(76) Inventor: Asha Nayak, Menlo Park, CA (US)

Correspondence Address: MEDTRONIC VASCULAR, INC.;IP LEGAL DEPARTMENT, 3576 UNOCAL PLACE, SANTA ROSA, CA, 95403, US

(21) Appl. No.: 11/619,576

(22) Filed: Jan. 3, 2007

Related U.S. Application Data
(63) Continuation-in-part of application No. 11/426,211, filed on Jun. 23, 2006.
(63) Continuation-in-part of application No. 11/426,219, filed on Jun. 23, 2006.
(60) Provisional application No. 60/693,749, filed on Jun. 23, 2005.
(60) Provisional application No. 60/743,686, filed on Mar. 23, 2006.
(60) Provisional application No. 60/693,749, filed on Jun. 23, 2005.
(60) Provisional application No. 60/743,686, filed on Mar. 23, 2006.

(51) Int. Cl. A61K 38/48; A61K 35/14

(52) U.S. Cl. 424/94.64; 424/532

(57) ABSTRACT

Methods and systems are disclosed for treating cardiac tissue by delivering a platelet composition which induces neovascularization in the cardiac tissue. The platelet composition can additionally contain structural materials and/or bioactive agents.

### CROSS REFERENCE TO RELATED APPLICATIONS

The present application is a continuation-in-part of U.S. patent application Ser. Nos. 11/426,211 and 11/426,219, both filed Jun. 23, 2006, both of which in turn claim priority under 35 U.S.C. §119(e) to U.S. Provisional Patent Application Nos. 60/693,749 filed Jun. 23, 2005 and 60/743,686 filed Mar. 23, 2006, the entire contents of which are incorporated by reference herein in their entirety.

### FIELD OF THE INVENTION

The present invention relates generally to systems and methods for treating injured cardiac tissue. Specifically, the present invention discloses compositions and methods for inducing neovascularization in the injured tissue.

### BACKGROUND OF THE INVENTION

The human heart wall consists of an inner layer of simple squamous epithelium, referred to as the endocardium, overlying a variably thick heart muscle or myocardium and is enveloped within a multi-layer tissue structure referred to as the pericardium. The innermost layer of the pericardium, referred to as the visceral pericardium or epicardium, covers the myocardium. The epicardium reflects outward at the origin of the aortic arch to form an outer tissue layer, referred to as the parietal pericardium, which is spaced from and forms an enclosed sac extending around the visceral pericardium of the ventricles and atria. An outermost layer of the pericardium, referred to as the fibrous pericardium, attaches the parietal pericardium to the sternum, the great vessels and the diaphragm so that the heart is confined within the middle mediastinum. Normally, the visceral pericardium and parietal pericardium lie in close contact with each other and are separated only by a thin layer of a serous pericardial fluid that enables friction free movement of the heart within the sac. The space between the visceral and parietal pericardia is referred to as the pericardial space. In common parlance, the visceral pericardium is usually referred to as the epicardium, and epicardium will be used hereafter. Similarly, the parietal pericardium is usually referred to as the pericardium, and pericardium will be used hereafter in reference to parietal pericardium.

Heart disease, including myocardial infarction (MI), is a leading cause of death and disability in human beings, particularly in the western world, most particularly among males. A variety of heart diseases can progress to heart failure by a common mechanism called remodeling. With remodeling, cardiac function progressively deteriorates, often leading to clinical heart failure and associated symptoms. Heart disease can in turn impair other physiological systems. Each year over 1.1 million Americans have a myocardial infarction (MI). Myocardial infarction can result in an acute depression in ventricular function and expansion of the infarcted tissue under stress. This triggers a cascading sequence of myocellular events known as remodeling. In many cases, this progressive myocardial infarct expansion and remodeling leads to deterioration in ventricular function and heart failure. Such ischemic cardiomyopathy is the leading cause of heart failure in the United States. It is the objective of the present invention to improve vascular supply to patients who have or are at high-risk of developing cardiac disease (such as cardiac ischemia). Acutely or chronically diseased cardiac tissue would benefit from increased blood supply. Studies have shown that even in the adult, normal repair mechanisms are elicited (e.g. those involving the recruitment of endogenous regenerative cells) following cardiac injury. Inadequate blood supply limits the survival of such cells and may prevent healing. Blood supply is required to bring necessary oxygen, nutrients, and blood components (cells, chemokines, etc.) to the injured region and to clear metabolic products. A treatment that improves blood supply to such a region is very likely to benefit the patient by facilitating greater recovery.

Cardiac tissue can be acutely or chronically ischemic. Severe ischemia resulting in cardiac cell death is referred to as infarction. Acute or chronic recovery may be improved by increasing vascular supply to or around the affected injured region.

A stenosed or blocked coronary artery is one example of heart disease. A completely or substantially blocked coronary artery can cause immediate, intermediate term, and/or long-term adverse effects. In the immediate term, a myocardial infarction can occur when a coronary artery becomes occluded and can no longer supply blood to the myocardial tissue, thereby resulting in myocardial cell death. When a myocardial infarction occurs, the myocardial tissue that is no longer receiving adequate blood flow dies and is eventually replaced by scar tissue.

Within seconds of a myocardial infarction, the under-perfused myocardial cells no longer contract, leading to abnormal wall motion, high wall stresses within and surrounding the infarct, and depressed ventricular function. The high stresses at the junction between the infarcted tissue and the normal tissue lead to expansion of the infarcted area and to remodeling of the heart over time. These high stresses injure the still viable myocardial cells and eventually depress their function. This results in an expansion of injury and dysfunctional tissue including and beyond the original myocardial infarct region.

According to the American Heart Association, in the year 2000 approximately 1,100,000 new myocardial infarctions occurred in the United States. For 650,000 patients this was their first myocardial infarction, while for the other 450,000 patients this was a recurrent event. Two hundred-twenty thousand people suffering Ml die before reaching the hospital. Within one year of the myocardial infarction, 25% of men and 38% of women die. Within 6 years, 22% of men and 46% of women develop heart failure, of which 67% are disabled. This is despite modern medical therapy.

The consequences of myocardial infarction are often severe and disabling. When a myocardial infarction occurs, the myocardial tissue that is no longer receiving adequate blood flow dies and is replaced with scar tissue. This infarcted tissue cannot contract during systole, and may actually undergo lengthening in systole and leads to an immediate depression in ventricular function. This abnormal motion of the infarcted tissue can cause delayed or abnormal conduction of electrical activity to the still surviving peri-infarct tissue (tissue at the junction between the normal tissue and the infarcted tissue) and also places extra structural stress on the peri-infarct tissue.

The zone receiving the reduced blood flow is known as an ischemic zone. Furthermore, the elevation of matrix metalloproteinases, reduction in tissue inhibitors of the matrix metalloproteinases (TIMPs), and consequent degradation of collagen may play an additional role in ischemic cardiomyopathy. To improve cardiac function in patients with ischemic cardiomyopathies, there is a need to re-establish blood flow to the ischemic zones.

In addition to immediate hemodynamic effects, the infarcted heart tissue undergoes three major processes: infarct expansion, infarct extension, and chamber remodeling. These factors individually and in combination contribute to the eventual dysfunction observed in the cardiac tissue remote from the site of the infarction

Infarct expansion is a fixed, permanent, disproportionate regional thinning and dilatation of tissue within the infarct zone. Infarct expansion occurs early after a myocardial infarction. The mechanism is slippage of the tissue layers.

Infarct extension is additional myocardial necrosis following myocardial infarction. Infarct extension results in an increase in total mass of infarcted tissue and the additional infarcted tissue may also undergo infarct expansion. Infarct extension occurs days after a myocardial infarction. The mechanism for infarct extension appears to be an imbalance in the blood supply to the peri-infarct tissue versus the increased oxygen demands on the tissue.

Remodeling is usually the progressive enlargement of the ventricle accompanied by a depression of ventricular function. Myocyte function in the cardiac tissue remote from the initial myocardial infarction becomes depressed. Remodeling occurs weeks to years after myocardial infarction. Such remodeling usually occurs on the left side of the heart. Where remodeling does occur on the right side of the heart, it can generally be linked to remodeling (or some other negative event) on the left side of the heart. Remodeling can occur independently in the right heart, albeit less often than the left. There are many potential mechanisms for remodeling, but it is generally believed that the high stress on peri-infarct tissue plays an important role. Due to variety of factors such as altered geometry, wall stresses are much higher than normal in the cardiac tissue surrounding the infarction.

The processes associated with infarct expansion and remodeling are believed to be the result of high stresses exerted at the junction between the infarcted tissue and the normal cardiac tissue (i.e., the peri-infarct region). In the absence of intervention, these high stresses will eventually kill or severely depress function in the adjacent cells. As a result, the peri-infarct region will therefore grow outwardly from the original infarct site over time. This resulting wave of dysfunctional tissue spreading out from the original myocardial infarct region greatly exacerbates the nature of the disease and can often progress into advanced stages of heart failure.

The treatments for myocardial infarction that are used currently, and that have been used in the past, are varied. Immediately after a myocardial infarction, preventing and treating ventricular fibrillation and stabilizing the hemodynamics are well-established therapies.

Ischemic heart disease can be acute or chronic. Mild disease results in inadequate blood supply during increased demand (e.g. during exertion). Severe disease results in inadequate blood supply even at rest. Both conditions would benefit from increased blood supply, as this would be expected to result in positive clinical sequellae. This may include any or all of increased exertional capacity, reduced symptoms, increased organ blood perfusion, improved cardiac output, and/or improved cardiac contractility.

Newer approaches include more aggressive efforts to restore patency to occluded vessels. This is accomplished through thrombolytic therapy or angioplasty and stents. Reopening the occluded artery (i.e. revascularization) within hours of initial occlusion can decrease tissue death, and thereby decrease the total magnitude of infarct expansion, extension, and thereby limit the stimulus for remodeling.

The direct or selective delivery of agents to cardiac tissue is often preferred over the systemic delivery of such agents for several reasons. One reason is the substantial expense and small amount of the medical agents available, for example, agents used for gene therapy. Another reason is the substantially greater concentration of such agents that can be delivered directly into cardiac tissue, compared with the dilute concentrations possible through systemic delivery. Yet another reason is that systemic administration is associated with systemic toxicity at doses required to achieve desired drug concentrations in the cardiac tissue.

One mode of delivering medical agents to cardiac tissue is by epicardial, direct injection into cardiac tissue during an open chest procedure. Another approach taken to delivery medical agents into cardiac tissue has been an intravascular approach. Catheters may be advanced through the vasculature and into the heart to inject materials into cardiac tissue from within the heart. Another approach is deliver materials into cardiac wall from within the chamber of the heart, an endocardial approach. Furthermore, additional therapies being developed for treating injured cardiac tissue include the injection of cells and/or other biologic agents into ischemic cardiac tissue or placement of cells and/or agents onto the ischemic tissue. One therapy for treating infarcted cardiac tissue includes the delivery of cells that are capable of maturing into actively contracting cardiac muscle cells or regenerating cardiac tissue. Examples of such cells include myocytes, myoblasts, mesenchymal stem cells, and pluripotent cells. Delivery of such cells into cardiac tissue is believed to be beneficial, particularly to prevent or treat heart failure.

It has been postulated that after acute or chronic injury to the heart, endogenous regenerative cells attempt to restore some or all function to the injured tissue. It is likely that the reduced blood flow and vascular supply to the injured region inhibits these recuperative mechanisms. The provision of more adequate perfusion may facilitate earlier, faster, and/or more complete recovery.

A focus remains on re-establishing blood flow to the ischemic zone which reduces symptoms and/or improves cardiac function. Re-establishing blood flow may be accomplished through stimulation of angiogenesis in which the body generates or expands blood supply to a particular region. Prior methods for re-establishing blood flow and rehabilitating the heart frequently involved invasive surgery such as bypass surgery or angioplasty. Other methods have used lasers to bore holes through the infarctions and ischemic zones to promote blood flow. These surgeries are complicated and dangerous. Therefore, a need exists for a safe less-invasive method for re-establishing blood flow.

None of these discussed methods specifically induce angiogenesis or neovascularization of the injured tissue. Establishment of an improved or normal blood supply within the injured tissue can prevent further deterioration and promote regeneration of the injured tissue. Such a treatment would be advantageous over previously used treatments. For these reasons, it is desirable to have an agent that could be delivered directly to cardiac tissue to induce neovascularization.

### SUMMARY OF THE INVENTION

The present invention provides biocompatible compositions for inducing neovascularization in injured cardiac tissue. Associated methods and systems for treating patients having cardiac tissue injuries are also provided.

In one embodiment of the present invention, a system for neovascularization of cardiac tissue is provided comprising a platelet composition and at least one delivery device for introducing the platelet composition into the cardiac tissue; wherein the platelet composition induces neovascularization in the cardiac tissue.

In another embodiment, the platelet composition is selected from the group consisting of platelet gel, platelet rich plasma and platelet poor plasma. In another embodiment, the platelet composition is autologous. In another embodiment, the platelet gel is formed from platelet poor plasma or platelet rich plasma and an activating agent. In another embodiment, the activating agent is thrombin. In another embodiment, the thrombin is selected from the group consisting of recombinant thrombin, human thrombin, animal thrombin, engineered thrombin and autologous thrombin. In another embodiment, the platelet gel is formed from platelet rich plasma or platelet poor plasma to thrombin at a ratio of between about 5:1 and about 25:1. In another embodiment, the ratio of platelet rich plasma or platelet poor plasma to thrombin is approximately 10:1.

In another embodiment, the platelet composition comprises platelet rich plasma without an exogenous source of thrombin. In yet another embodiment, the platelet composition is delivered to the treatment site and forms a gel within the cardiac tissue at the treatment site.

In another embodiment of the present invention, the system further comprises a structural material selected from the group consisting of collagen, biocompatible polymers, alginates, synthetic/natural compounds, fibrinogen, silk-elastin polymers, hydrogels, and dental composite material. In another embodiment, the structural material forms a solid or a gel as a result of physical or chemical cross-linking or activation, wherein the activation is selected from the group consisting of enzymatic, chemical, thermal or light activation of the composition.

In another embodiment of the present invention, the platelet composition further comprises a bioactive agent. In another embodiment, the bioactive agent is selected from the group consisting of pharmaceutically active compounds, hormones, growth factors, enzymes, DNA, RNA, siRNA, viruses, proteins, lipids, polymers, hyaluronic acid, antibodies, antibiotics, anti-inflammatory agents, anti-sense nucleotides and transforming nucleic acids, and combinations thereof. In another embodiment, the platelet composition further comprises a contrast agent.

In another embodiment of the present invention, the platelet composition is provided to the injured cardiac tissue between about 1 hour and about 1 year after injury occurs to the cardiac tissue. In another embodiment, the platelet composition is provided in approximately 1 to 20 injections. In another embodiment, the injections are provided sequentially. In another embodiment, the injections are provided approximately simultaneously. In another embodiment, the platelet composition comprises a total injection volume up to about 15 mL. In another embodiment, the platelet composition comprises an injection volume up to about 1100 microliters per injection.

In an embodiment of the present invention, the platelet composition is injected into the cardiac tissue at an angle orthogonal or oblique to the tissue surface. In another embodiment, the cardiac tissue is selected from the group consisting of sub-endocardial, sub-epicardial and intra-myocardial sites. In another embodiment, the platelet composition is injected into the cardiac tissue at a depth approximately midway through the thickness of the myocardium.

In another embodiment of the present invention, the delivery device is an injection catheter selected from the group consisting of an endocardial injection catheter, a transvascular injection catheter and an epicardial injection catheter.

In an embodiment of the present invention, the platelet composition is provided to the treatment site during an injurious event or after an injurious event has occurred. In another embodiment, the treatment site is selected from the group consisting of the injured area, the peri-injury area and the healthy tissue surrounding the injured area.

In one embodiment of the present invention, a method of inducing neovascularization in cardiac tissue is provided comprising a platelet composition at a treatment site in cardiac tissue wherein said platelet composition induces neovascularization in the cardiac tissue. In another embodiment, the platelet composition is selected from the group consisting of platelet gel, platelet rich plasma and platelet poor plasma. In another embodiment, the platelet composition is autologous.

### BRIEF DESCRIPTION OF THE DRAWINGS

FIG. 1 is a drawing of a normal, healthy heart.

FIG. 2 is a drawing of a heart with a region of injured myocardium.

FIG. 3 is an enlarged view of the injured myocardium depicted in FIG. 2.

FIG. 4 is a cross-sectional depiction of the heart shown in FIG. 1.

FIG. 5 is a cross-sectional depiction of a heart showing a region of injured and remodeled cardiac tissue on the wall of the left ventricle. Eligible injured cardiac tissue can be of different thicknesses and geometries. One example is shown with a mildly thinned and dilated (aneurismal) wall.

FIG. 6 depicts a needle being used to deliver a composition to the cardiac wall according to an embodiment of the current invention

FIG. 7 depicts a needle being used to deliver a composition to the cardiac wall according to an embodiment of the current invention.

FIG. 8 is a block diagram showing the steps of treating cardiac tissue according to the teachings of the current invention.

FIG. 9 schematically depicts delivery of a composition into the heart according to one embodiment of the present invention.

FIG. 10 schematically depicts a detailed view of delivery of a composition into cardiac tissue according to another embodiment of the present invention.

FIG. 11 schematically depicts the migration of a composition within the myocardial tissue after delivery according to an embodiment of the present invention.

FIG. 12 schematically depicts an epicardial approach to delivery of compositions to cardiac tissue according to the teachings of the present invention.

FIG. 13A-B schematically depicts an endocardial approach to delivery of compositions to cardiac tissue according to the teachings of the present invention. FIG. 13A depicts an anterograde endocardial approach through the venous system and FIG. 13B depicts a retrograde endocardial approach through the arterial system.

FIG. 14A-B schematically depicts a transvascular approach to delivery of compositions to cardiac tissue according to the teachings of the present invention. FIG. 14A depicts a venous approach and FIG. 14B depicts an arterial approach through the coronary artery.

FIG. 15 depicts a flow diagram of the system of the present invention.

FIG. 16 depicts a photomicrograph of infarcted myocardium eight weeks after injection with autologous platelet gel (platelet rich plasma and bovine thrombin at 10:1 ratio) one hour after infarction according to the teachings of the present invention. Many blood vessels (arrow A) are observed within a region of infarcted tissue (arrow C). These vessels are carrying red blood cells (arrow B).

FIG. 17 depicts a higher magnification photomicrograph of infarcted myocardium eight weeks after injection with autologous platelet gel (platelet rich plasma and bovine thrombin at 10:1 ratio) one hour after infarction according to the teachings of the present invention. Many blood vessels (arrow A) are observed within a region of infarcted tissue (arrow C). These vessels are carrying red blood cells (arrow B).

### DEFINITION OF TERMS

Generally, all technical terms or phrases appearing herein are used as one skilled in the art would understand to be their ordinary meaning. For the convenience of the reader, however, selected terms are more specifically defined as follows.

Angiogenesis: As used herein, “angiogenesis” refers to a physiologic process involving the growth of new blood vessels from pre-existing blood vessels.

Bioactive agent: As used herein, “bioactive agent” includes therapeutic agents and drugs and includes pharmaceutically active compounds, hormones, growth factors, enzymes, DNA, RNA, siRNA, viruses, proteins, lipids, polymers, hyaluronic acid, antibodies, antibiotics, anti-inflammatory agents, anti-sense nucleotides and transforming nucleic acids, inhibitors of compounds implicated in remodeling (e.g., inhibitors of angiotensin 11, angiotensin converting enzyme, atrial natriuretic peptide, aldosterone, renin, norepinephrine, epinephrine, endothelin, etc.) and combinations thereof.

Chamber remodeling: As used herein, “chamber remodeling” refers to remodeling of the atria or ventricles. “Remodeling” refers to a series of events (which may include changes in gene expression, molecular, cellular and interstitial changes) that result in changes in size, shape and function of cardiac tissue following stress or injury. Remodeling may occur after myocardial infarction (MI), pressure overload (e.g., aortic stenosis, hypertension), volume overload (e.g., valvular regurgitation), inflammatory heart disease (e.g., myocarditis), or in idiopathic cases (e.g., idiopathic dilated cardiomyopathy). Remodeling is often pathologic, resulting in progressively worsening cardiac function and ultimately a failing heart. Pathologic remodeling as described above will be referred to as remodeling in this disclosure.

Cardiac tissue injury: As used herein, “cardiac tissue injury” refers to any area of abnormal tissue in the heart caused by a disease, disorder or injury and includes damage to the epicardium, endocardium, and/ or myocardium. Non-limiting examples of causes of cardiac tissue injury include acute or chronic stress (systemic hypertension, pulmonary hypertension, valve dysfunction, etc.), coronary artery disease, ischemia or infarction, inflammatory disease and cardiomyopathies. Cardiac tissue injury most often involves injury to the myocardium and therefore, for the purposes of this disclosure, myocardial injury is equivalent to cardiac tissue injury. Furthermore, there are occasions when the injury is acute, such as in an acute myocardial infarction, where the injury may be referred to as an injurious event. Injured cardiac tissue includes tissue that is ischemic, infarcted, or otherwise focally or diffusely diseased.

Composition: As used herein, “composition” refers to an injectate, substance or a combination of substances which can be delivered into a tissue and are used interchangeably herein. Exemplary compositions include, but are not limited to, platelet gel, autologous platelet gel, platelet rich plasma and platelet poor plasma, with and without the addition of bioactive agents, structural materials, etc.

Delivery: As used here, “delivery” refers to providing a composition to a treatment site in an injured tissue through any method appropriate to deliver the functional composition to the treatment site. Non-limiting examples of delivery methods include direct injection at the treatment site, direct topical application at the treatment site, percutaneous delivery for injection, percutaneous delivery for topical application, and other delivery methods well known to persons of ordinary skill in the art.

Injury area: As used herein, “injury area” refers to the injured tissue. The “peri-injury area” refers to the tissue immediately adjacent to the injured tissue. That is, the tissue at the junction between the injured tissue and the normal tissue.

Injured tissue: As used herein, “injured tissue” refers to tissue injured by trauma, ischemic tissue, infarcted tissue or tissue damaged by any means which results in interruption of normal blood flow to the tissue. Related to the heart, “injured tissue” includes tissue undergoing any of the changes described under “cardiac tissue injury”.

Neovascularization: As used herein, “neovascularization” refers to the formation of functional vascular networks that may be perfused by blood or blood components. Neovascularization includes angiogenesis, budding angiogenesis, intussuceptive angiogenesis, sprouting angiogenesis, therapeutic angiogenesis and vasculogenesis.

Percutaneous: As used herein, the term “percutaneous” refers to any penetration through the skin of the patient, whether in the form of a small cut, incision, hole, cannula, tubular access sleeve or port or the like. A percutaneous penetration may be made in an interstitial space between the ribs of the patient or it may be made elsewhere, such as the groin area of a patient.

Structural support: As used herein, the term “structural support” refers to mechanical reinforcement providing resistance against the stresses and maladaptive processes of remodeling.

Vasculogenesis: As used herein, the term “vasculogenesis” refers to blood vessels formation by de novo production of endothelial cells, a process that occurs during development and also in adulthood (e.g. after trauma or after cardiac injury).

### DETAILED DESCRIPTION

The present invention provides biocompatible compositions for inducing neovascularization in injured cardiac tissue. Associated methods and systems for treating patients having cardiac tissue injuries are also provided.

After an injury, such as an ischemic insult, blood supply to the tissue is often insufficient to maintain the remaining healthy tissue. The persistently ischemic tissue dies and neighboring tissue is at increased risk of ischemia. This process results in the growth of the area of ischemia with time. Increasing the blood supply to the damaged or neighboring tissue can prevent further ischemia as well as chamber remodeling.

In embodiments of the present invention, compositions and methods are provided for inducing angiogenesis in cardiac tissue by injecting a platelet composition directly into the injured or surrounding heart tissue. In one embodiment, the platelet composition induces neovascularization. In another embodiment, the platelet composition induces neovascularization to regenerate injured tissue. In another embodiment, the induced neovascularization prevents or reverses chamber remodeling. In yet another embodiment, the platelet composition induces neovascularization to promote regeneration of cardiac tissue or function.

Neovascularization refers to the development of new blood vessels from endothelial precursor cells by any means, such as by vasculogenesis, angiogenesis, or the formation of new blood vessels from endothelial precursor cells that link to existing blood vessels. Angiogenesis is the process by which new blood vessels grow from the endothelium of existing blood vessels in a developed animal. Endothelial precursor cells circulate in the blood and selectively migrate, or “home,” to sites of active neovascularization (see U.S. Pat. No. 5,980,887, Isner et al., the contents of which are incorporated herein by reference in their entirety).

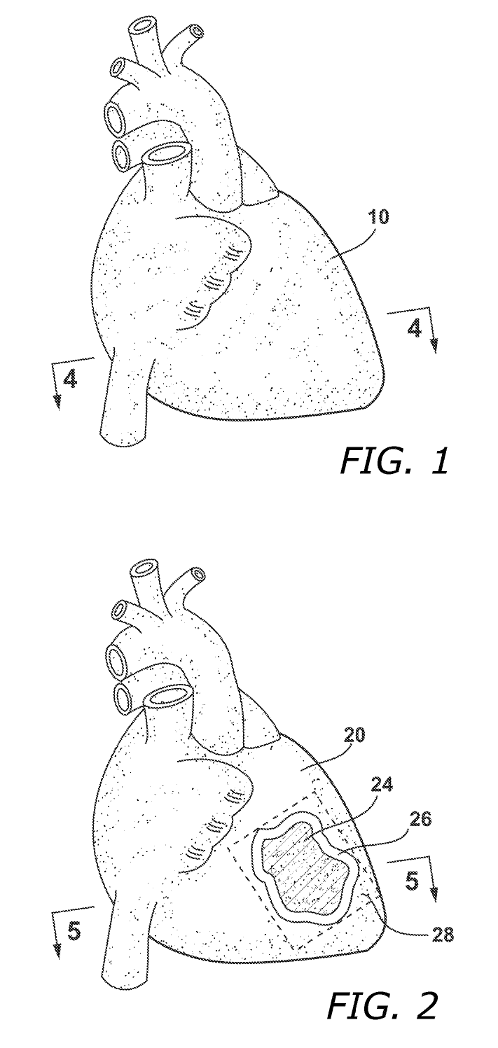

The present invention will now be described in detail below by reference to the drawings, wherein like numbers refer to like structures. Referring to FIGS. 1 and 4, there can be seen depictions of a normal heart 10. The cross-sectional view in FIG. 4 shows the right ventricle 44 and the left ventricle 42 of a normal heart that has not undergone chamber remodeling.

FIG. 2 depicts a heart 20 having an ischemic or infarcted region 24, and a peri-infarct region 26 that is surrounded by healthy non-ischemic myocardium 28. When an infarction occurs, the cardiac tissue that is no longer receiving adequate blood flow dies and is replaced with scar tissue. Triggered by injury, a cascade of events (remodeling) cause the walls to thin, dilate, and ultimately fail. Inadequate blood flow in injured tissue prevents healing, in which endogenous cells and mechanisms may otherwise lead to repopulation and repair of the injured tissue.

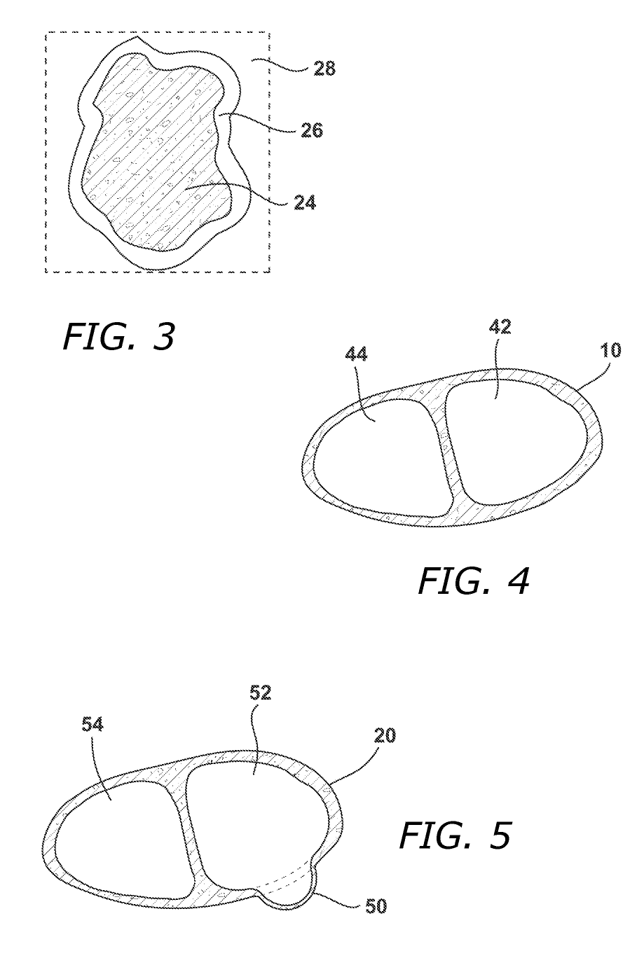

FIG. 3 is an enlarged view of the area bordered by dotted lines in FIG. 2. FIGS. 2 and 3 depict an area of myocardium 24 that has undergone some kind of ischemic insult such as an MI or other injury. If necrosis has occurred, that portion of myocardium that has experienced necrosis will be totally infarcted. The area immediately surrounding the ischemic/infarcted area 26 is known as the peri-infarct area and is surrounded by healthy myocardium 28. As discussed above, the peri-infarct area 26 may have experienced some level of ischemic activity but the blood supply has not yet been interrupted to the same extent as that of the ischemic/infarcted area 24.

Remodeling is usually the progressive enlargement of the ventricle accompanied by a deterioration in ventricular function, and it can occur weeks to years after myocardial infarction. There are many potential mechanisms for remodeling, but it is generally believed that the high stress on peri-infarct tissue plays an important role. Due to altered geometry, wall stresses are much higher than normal in the myocardial tissue surrounding the infarction. FIG. 5 is a cross sectional view of the heart shown in FIG. 2. FIG. 5 shows a right ventricle 54 and a left ventricle 52 having an area 50 of a left ventricle that has undergone remodeling. As can be seen in the figure, the heart walls are thinner in the expanded area 50.

A limited amount of remodeling can be beneficial for the patient and occurs mainly in two contexts. The first is termed “physiologic remodeling” which occurs in some high-performance athletes as an adaptive response to above-normal demands on the heart. The compensatory changes in cardiac geometry and function in the physiologically remodeled heart render it better able to perform in a high-performance environment. The second context is during the earliest stages of post-injury remodeling. Sometimes, the initial phase of this remodeling can actually be adaptive and protective. If to a limited degree, some cellular rearrangement within the cardiac wall and increased chamber volume, can preserve or even augment cardiac output. These changes can be beneficial. However, most often, this progresses beyond what is adaptive to “pathologic remodeling”, in which further changes in wall composition and geometry result in a progressively dysfunctional chamber and eventually a failing heart. “Pathological remodeling” as described above will be referred to in this disclosure as “remodeling.”

Although the details remain under investigation, the mechanism of remodeling appears to involve a cascade of events. As myocytes stretch under mechanical stress, local activity of several molecules increases (e.g. norepinephrine, angiotensin, endothelin, and others). These molecules stimulate expression of specific proteins and lead to the hypertrophy of existing myocytes. This causes further deterioration in cardiac function (e.g. added mechanical stress) and increased neurohormonal activation. Some of the released factors further stimulate local collagen synthesis which leads to fibrosis and scarring of the affected area. These changes are often beyond compensatory, and lead to a progressively failing heart.

In the absence of adequate blood flow in the injured region, endogenous repair mechanisms are not able to restore cardiac tissue or function. Endogenous cells have been demonstrated to “home” to injured tissue, even in the adult heart, but blood flow limitations may prevent them from taking residence and promoting healing.

Progressive deterioration in heart function can occur initially in the absence of symptoms. Eventually, however, symptoms of clinical heart failure develop, such as shortness of breath, swelling, difficulty breathing in the supine position, arrhythmias, organ failure, etc. It is important to note that even patients with asymptomatic cardiac dysfunction and milder forms of heart failure are at increased risk of sudden cardiac death. Thus, there is great incentive to treat this disease process early and effectively.

Measures to assess cardiac remodeling include cardiac size, cardiac shape, cardiac mass, ejection fraction, end-diastolic and end-systolic volumes, and peak force of contraction. Left ventricular volume (especially left ventricular end systolic volume) is the best predictor of mortality in humans after myocardial infarction.

Pharmaceutical therapies, which provide angiotensin-converting-enzyme inhibition (e.g., captopril, enalapril) and beta-adrenergic blockade (e.g., carvedilol, metoprolol, propranolol, timolol) have been shown to slow certain parameters of cardiac remodeling. These therapies are intended to reduce the body's remodeling response to injurious or mechanically stressful stimuli and have been shown in clinical trials to reduce mortality and morbidity in myocardial infarction and heart failure patients. Other therapies, such as anti-hypertensive agents, have been used to reduce chronic loads placed on the heart which can trigger or worsen pathologic remodeling. Despite the use of the aforementioned drugs, remodeling remains at best, a process that is partially treatable. Furthermore, none of these agents induce neovascularization in the injured tissue as a means of preventing further cardiac damage or restoring cardiac tissue or function.

As described further below, embodiments of the present invention address cardiac injury and remodeling by injecting a composition into the cardiac wall to induce neovascularization and thus prevent remodeling. The injected composition may occupy some of the interstitial space between the cells of an area of the cardiac wall and provide structural reinforcement of the tissue in addition to inducing neovascularization. The present invention contemplates providing neovascularization to any cardiac wall site and includes both the atria and ventricles.

The injected platelet composition may be a substance that can provide some level of structural support as well as the desired neovascularization in the tissue. Substances that can provide both structural reinforcement of the tissue and stimulate neovascularization are included in the platelet compositions disclosed herein. For the purpose of this document, the term “platelet gel” refers to platelet compositions which are administered with an activating agent and may provide both structural reinforcement of the tissue and biological therapy such as neovascularization. Furthermore, the platelet composition can refer to platelet rich or platelet poor plasma that is administered without an activating agent. Platelet compositions such as platelet rich and platelet poor plasma can additionally be activated by tissue thrombin in situ to provide both structural support and neovascularization. Exemplary, non-limiting platelet compositions include platelet gel, autologous platelet gel, platelet rich plasma, and platelet poor plasma.

The neovascularization-inducing platelet compositions of the present invention can be administered with other compositions capable of providing structural support including, but not limited to, collagen, cyanoacrylate, adhesives that cure with injection into tissue, liquids that solidify or gel after injection into tissue, suture material, agar, gelatin, light-activated dental composite, other dental composites, silk-elastin polymers, Matrigel® (BD Biosciences), hydrogels and other suitable biopolymers. Such compositions can include single or multi-component compounds. These compositions can include agents that are delivered as a liquid and then gel or harden to a solid after delivery. The hardening/gelling can be triggered by temperature, pH, proteins, or other environmental factors inherent in or created within the target tissue. These platelet compositions can be injected separately or in combination with each other and/or platelet gel. Additionally the compositions or combinations thereof can include other additives. Some of these compositions and/or additives are further described below.

The platelet compositions of the current invention can be fortified with or comprised wholly of a biocompatible liquid that solidifies and/or cross-links in situ to render a structurally supportive structure on delivery into the cardiac wall. Other embodiments of the platelet compositions of the current invention may include synthetic or naturally-occurring materials and/or non-degradable or biodegradable materials to provide strength, for example. In one embodiment, the structural material includes cyanoacrylate or silk-elastin protein polymers.

The platelet compositions of various embodiments of the current invention can include additives, such as fibrinogen, to increase the structural strength of the cardiac wall. The fibrinogen can be autologous, allogeneic, recombinant, human, engineered, or purified from animal sources. At least one embodiment includes elastin to increase the elasticity of the treated cardiac wall. The compositions may be delivered as a liquid (without cross-linking or solidifying components) such that the key soluble factors are trapped in the target tissue (physically or by binding to sites in the tissue) without providing an inherent structural component. Alternatively, the compositions may be delivered with one or more structural materials to provide additional structural support to the tissue.

The present invention may be practiced using substances containing synthetic biodegradable materials that provide strength for a specified time interval after delivery, and then resorb. Such materials include genetically-engineered or modified compounds such as collagen or fibrin. Naturally-occurring materials, such as, but not limited to, cartilage, bone or bone components, gelatin, collagen, glycosaminoglycans, starches, polysaccharides, or any other material that provide strength for a specified time interval after delivery, and then resorbs, may also be used.

Other embodiments of the present invention may include a combination of any of a variety of compounds that can create the desired local effect of tissue bulking. Components that cause local edema, thickening of the tissue, structural reinforcement of the tissue, or any other effect that prevents remodeling are included in this invention. Such compounds include ground-up suture material to create edema and hydrogels for structural reinforcement of the tissue. These materials may be added to PRP or PRP + thrombin.

If a desired effect is structural reinforcement of the tissue, biodegradable micro-particles between 50-100 μm in size (at the widest point of the particle), such that they are small enough for needle injection but too large to fit into capillaries and venules, may be added to the platelet composition. The micro-particles may be impregnated with a drug that elutes as the particles degrade. In one embodiment of the present invention, micro-particles alone are delivered to the cardiac tissue by injection into the coronary sinus. Based on their size characteristics, they are expected to lodge in the tissue and provide structural reinforcement of the tissue. The micro-particles used may have a glass transition temperature (Tg) ≧37° C., so they would gel over days after insertion. The injected micro-particles would provide “mass” and volume for immediate structural reinforcement of the tissue, but soften to gel to become a single member over time.

Embodiments of the platelet compositions of the present invention may include polymers that can covalently bind directly to one or more proteins located on the surface of one or more cell types so as to retain the polymers at the local site of injection. In one embodiment, polymers that can covalently bind to the primary amine groups (−NH₃) of proteins may be used.

Before any composition is injected into a heart having a region of injured tissue, to induce neovascularization or to provide structural reinforcement of the tissue to the cardiac wall, the location and extent of the injured region is identified. Multiple technologies and approaches are available for the clinician to identify and assess normal, injured-non-viable, and injured-viable cardiac tissue. These include, but are not limited to, visual inspection during open chest surgical procedures, localized blood flow determinations, local electrical and structural activity, nuclear cardiology, echocardiography, echocardiographic stress test, coronary angiography, magnetic resonance imaging (MRI), computerized tomography (CT) scans, and ventriculography.

In one embodiment of the invention, the platelet compositions are prepared by using the Medtronic Magellan® Platelet Separator. Anticoagulated whole blood is prepared by combining an anticoagulant with whole blood freshly removed from the subject. The Magellan® device is used to then extract platelet rich plasma (PRP) and platelet poor plasma (PPP) from the sample of anticoagulated whole blood. Platelet gel is prepared by combining the resulting PRP or PPP with an activator. In one embodiment the activator is bovine thrombin which has been reconstituted to 1000 Units/milliliter in 10% calcium chloride solution. In another embodiment, PRP is combined in an approximately 10:1 ratio with bovine thrombin.

In one embodiment of the present invention, the composition is a platelet gel that is made using a PRP to thrombin ratio of about 10:1. Another embodiment uses a PRP to thrombin ratio of about 11:1. Other embodiments of the present invention have ratios of PRP to thrombin of about 5:1 to about 25:1. In another embodiment, the ratio of PRP to thrombin is about 7:1 to about 20:1. In another embodiment, the ratio of PRP to thrombin is about 9:1 to about 15:1. In another embodiment, the ration of PRP to thrombin is about 10:1 to about 12:1. In at least one embodiment, no thrombin is included and PRP is injected into the cardiac tissue alone. Other embodiments of the present invention include multiple components of the composition in ratios needed to achieve or optimize the desired effect.

The PRP contains a high concentration of platelets that can aggregate during gelling, as well as release cytokines, growth factors or enzymes following activation. Some of the many factors released by the platelets and the white blood cells that constitute the PRP include platelet-derived growth factor (PDGF), platelet-derived epidermal growth factor (PDEGF), fibroblast growth factor (FGF), transforming growth factor-beta (TGF-β) and platelet-derived angiogenesis growth factor (PDAF). These factors have been implicated in wound healing by increasing the rate of collagen secretion, vascular in-growth and fibroblast proliferation.

Once the location, size and shape of the injured region are identified, the clinician can access and begin injecting the cardiac wall with the platelet compositions. In one embodiment, the platelet composition comprises PRP and thrombin. In another embodiment, the platelet composition comprises PRP alone. In another embodiment, the platelet composition comprises PPP and thrombin. In yet another embodiment, the platelet composition comprises PPP alone. The components of the platelet composition may be derived from humans, and/or animals, and/or recombinant sources. The components may also be artificially produced. The components for platelet composition can be categorized as autologous, or non-autologous, and the non-autologous components can be further categorized as described above (i.e., animal, recombinant, engineered, allogeneic human, etc.). Autologous platelet gel (APG) refers to a composition made from autologous PRP or autologous PPP and an autologous or non-autologous activator. One advantage in using autologous and/or recombinant components in the injected compositions is that it reduces the recipient's risk of an inflammatory response or exposure to infectious and foreign agents.

Additionally, the platelet composition can include one or more bioactive agents to induce healing or regeneration of damaged cardiac tissue. Suitable bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof. The platelet composition may also include cellular additives such as stem cells, leukocytes, red blood cells, cultured cardiac cells, or other differentiated or undifferentiated cells.

Furthermore, the platelet compositions of the present invention can include a contrast agent for detection by X-rays, magnetic resonance imaging (MRI) or ultrasound. Suitable contrast agents are known to persons of ordinary skill in the art and include, but are not limited to, radiopaque agents, echogenic agents and paramagnetic agents. A contrast agent may be used in the composition of some embodiments for visual confirmation of injection success. Examples of such contrast agents include, but are not limited to, X-ray contrast (e.g., IsoVue or other contrast agents having a high X-ray attenuation coefficient), MRI contrast (e.g., gadolinium or other contrast agents detectable as signal or signal-void by MRI), and ultrasound contrast (echogenic or echo-opaque compounds).

When the PRP and thrombin are injected such that they mix to form platelet gel in the cardiac tissue (see description of delivery devices below) they will gel in the tissue. Several embodiments of the present invention provide accelerated gel times. The gelling time in situ can be accelerated by applying local heat to the injection site via a delivery catheter or other instrument, increasing the thrombin concentration, or combining the PRP and thrombin in a mixing chamber and injecting the mixture into the cardiac tissue after the mixture has begun gelling. This description also applies for other multi-component compositions, where the components gel, cross-link and/or polymerize after being mixed together.

As described in further detail in the Examples, the platelet compositions of the present invention have been injected into the injured cardiac wall of test subjects (sheep and pigs). The experiments indicate that injections of PRP and thrombin are safe and well tolerated when made into infarcted or non-infarcted tissue, and that they can be performed safely as early as 1 hr post-MI. Controlled injections were possible with or without a cardiac stabilization device, and it was possible to make the injections without exogenous cardiac pacing. Injections were made both orthogonally and obliquely to the cardiac surface at intervals of 0.5 to 2.5 cm. A plurality of injections can be made per heart without safety problems. The total injectate volume can be as high as 15.0 mL, and the volume of individual injections can be as high as 1100 μl per injection site.

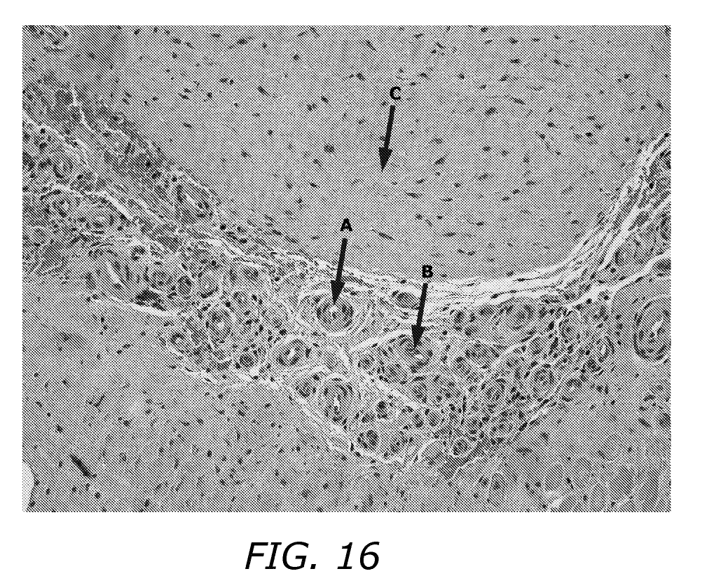

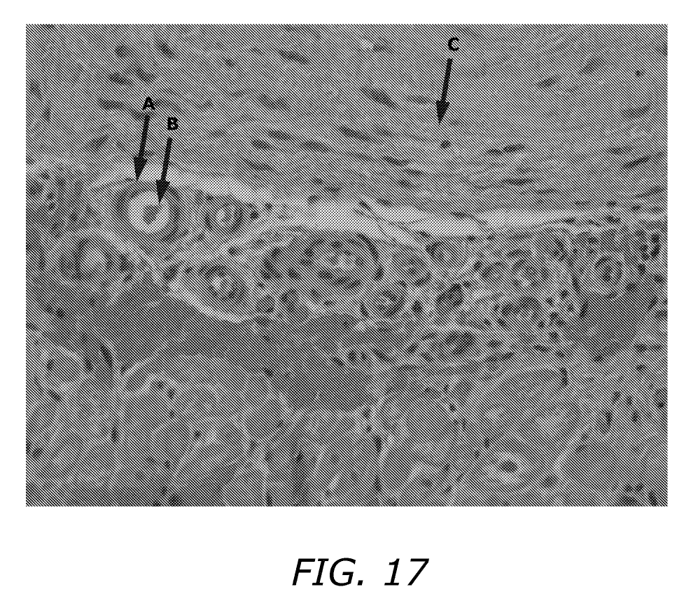

Furthermore, autologous platelet gel administration following cardiac injury partially or fully reverses detrimental acute effects of infarction on the ejection fraction (EF), and can augment EF towards or above pre-infarct levels. In a surprising result, autologous platelet gel administration following myocardial injury into ischemic tissue stimulated neovascularization of the injured tissue (FIG. 16-17). This vascularization was not observed in infarcted animals not receiving platelet gel therapy. All or a subset of the components of platelet gel (PRP or PPP components with or without thrombin) may be used to generate such an effect.

In order to practice the present invention and deliver a platelet composition to target sites within the cardiac wall, a clinician may use one of a variety of access techniques. These include surgical (sternotomy, thoracotomy, mini-thoracotomy, sub-xiphoid) approaches and percutaneous (transvascular and endocardial) approaches. Once access has been obtained, the composition may be delivered via epicardial, endocardial, or transvascular approaches. The platelet composition may be delivered to the cardiac wall tissue in one or more locations. This includes intra-myocardial, sub-endocardial, and/or sub-epicardial administration.

One method to predictably deliver platelet compositions into such a moving target tissue is to time injections specifically for delivery during a select portion of the cardiac cycle. In one embodiment of the present invention, one or more electrodes may be used as stimulation electrodes, e.g., to pace the heart during delivery of composition. In this way, the cardiac cycle is made to be predictable and injection can be timed and synchronized to it. In fact, the beat-to-beat period can be artificially lengthened so as to permit complete delivery during a specific (and relatively) stationary phase of the cardiac cycle. In one embodiment, the delivery device includes one or more stimulation and/or sensing electrodes. In one embodiment of the present invention, sensors may be used to sense contractions of the heart, thereby allowing the delivery of composition to be timed with cardiac contractions. For example, it may be desirable to deliver one or more components of the platelet composition between contractions of the heart.

Regardless of the method used to access a heart having a region of injured cardiac tissue or stabilize the heart, the delivery devices used may need to be capable of injecting multiple components separately into the cardiac wall. One embodiment of the current invention enables repeated injection by a single device. This may be achieved by a proximal one-hand trigger that enables predictable delivery of a determinable (e.g., dial-in) dose of a single- or multiple-constituent composition in a determinable ratio. A different embodiment of the current invention utilizes delivery devices having dual lumen needles/delivery catheters, and at least one other embodiment uses delivery devices having three or more lumen needles/delivery catheters. The lumens in the needles/delivery catheters can be in a coaxial configuration or a biaxial configuration.

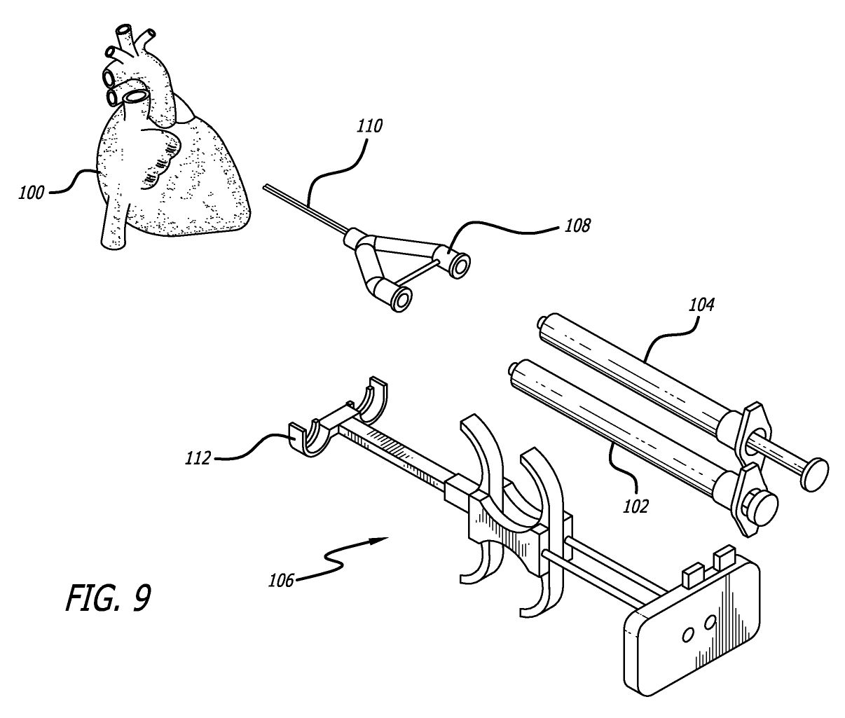

At least one embodiment of the present invention includes two or more side-by-side syringes for one-handed injection of the multiple composition components. In one embodiment, the device of FIG. 9 is used to inject a multi-component composition into an injured heart 100. In the embodiment of FIG. 9, two components of the composition of the present invention are housed separately in syringes 102 and 104. Syringes 102 and 104 are disposed in cradle 112 within a handle assembly 106 to allow one-handed injection of the composition. An adapter 108 couples to the syringes 102 and 104 to a biaxial needle 110. Biaxial needle 110 allows the delivery of two components of a composition, in a non-limiting example, PRP and thrombin, to a treatment site in heart 100.

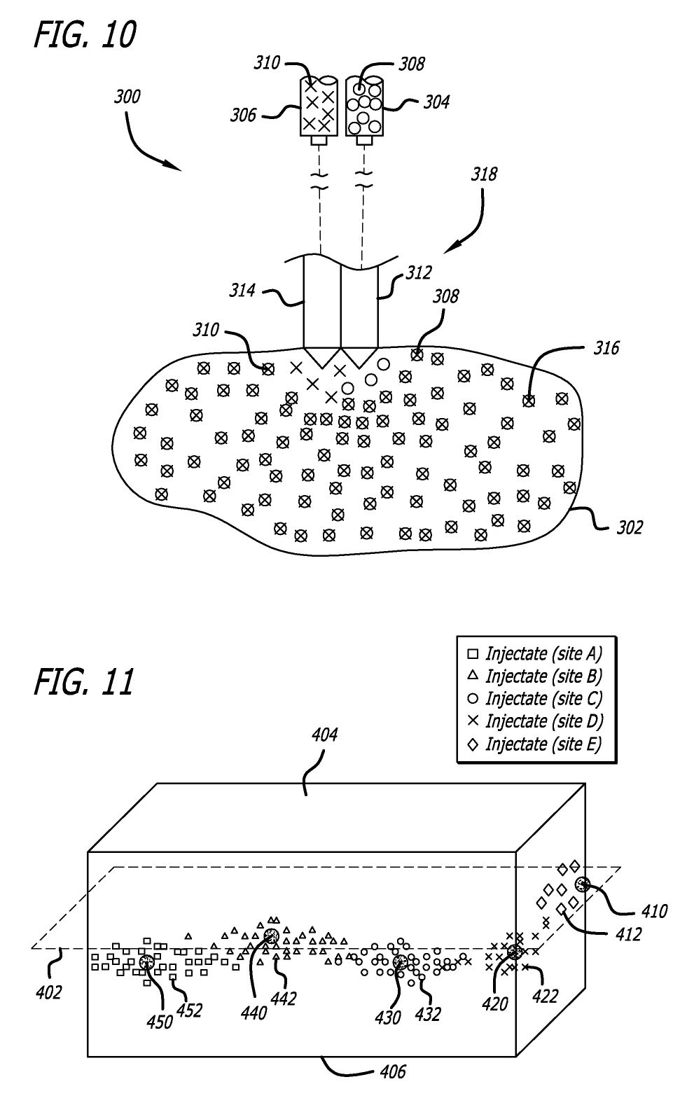

FIG. 10 represents an enlarged view of the injection of a two-component composition according to the present invention using a biaxial injection needle containing delivery device 300. Component 310 is held in reservoir or syringe 306 and component 308 is held in reservoir or syringe 304. Components 310 and 308 are caused to pass into biaxial needle 318 comprising needle lumen 314 for injection of component 310 and needle lumen 312 for injection of component 308. Components 310 and 308 are injected into the treatment site 302 simultaneously and the two components combine to form composition 316. Immediately after injection, components 310 and 308, and to a certain extent composition 316 diffuse through the tissue at treatment site 302. The components and compositions have been observed to diffuse up to two centimeters in cardiac tissue (see also FIG. 11).

The delivery system may delivery the components of the composition in a prescribed ratio. This ratio may be pre-set (and fixed) or dialable (and dynamic). One embodiment of the present invention utilizes separate gears or levers (with gear-ratio or lever-ratio that are settable) to enable delivery of multiple compounds in different ratios without generating a pressure gradient between syringes. Other multi component delivery devices of the current invention include lumens of different caliber to allow for pre-determined ratio of each component. Some multi-component delivery devices of the current invention include lumens of different lengths, such that one component is released more distally than another. Still other devices incorporate one or more mixing chambers in the device. At least one embodiment of delivery devices of the current invention includes single lumen needle/catheters that are used for serial delivery of multiple components (one after another).

Several embodiments of delivery devices can be placed in a vessel neighboring the target treatment site and used to deliver platelet compositions to the cardiac wall by piercing through the vessel wall and navigating to the desired location with the needle-tip or a microcatheter that is contained in the needle. The catheter or needle may contain a local imaging system for identifying the target area and proper positioning of the delivery device. The device may include one or more needles having a closed distal tip and one or more side openings for directing a substance substantially laterally from the distal tip into the cardiac wall. Preferably, the needle has a sufficiently small gauge diameter such that the needle track in the cardiac wall is substantially self-sealing to prevent escape of the composition upon removal of the needle. Recent data (obtained in the context of epicardial delivery) demonstrated hemostasis in vivo when platelet gel was injected through even a large 18 gauge injection needle. This result could be attributable to the rapid coagulation achieved by the components injected and the inherent hemostatic properties of platelet gel. In another embodiment, the needle gauge is smaller than 18 gauge. In one embodiment, the needle gauge is 26 gauge.

Alternatively, the delivery assembly may include one or more needles having a plurality of lumens that extend between a multiple line manifold on the proximal end to adjacent outlet ports. A multi-lumen needle assembly may allow components of a substance to be independently injected, thereby allowing the components to react with one another following delivery within the selected tissue region, as described herein.

In one embodiment, a multi-lumen needle assembly may allow two components of a composition to be simultaneously, independently injected, which may then react with one another once within the selected tissue region, as described herein. In another embodiment having a multi lumen needle assembly, the lumens empty into a mixing chamber located near the distal tip of the needle and the components of the injected substance are mixed with each other immediately prior to being injected into the selected tissue region.

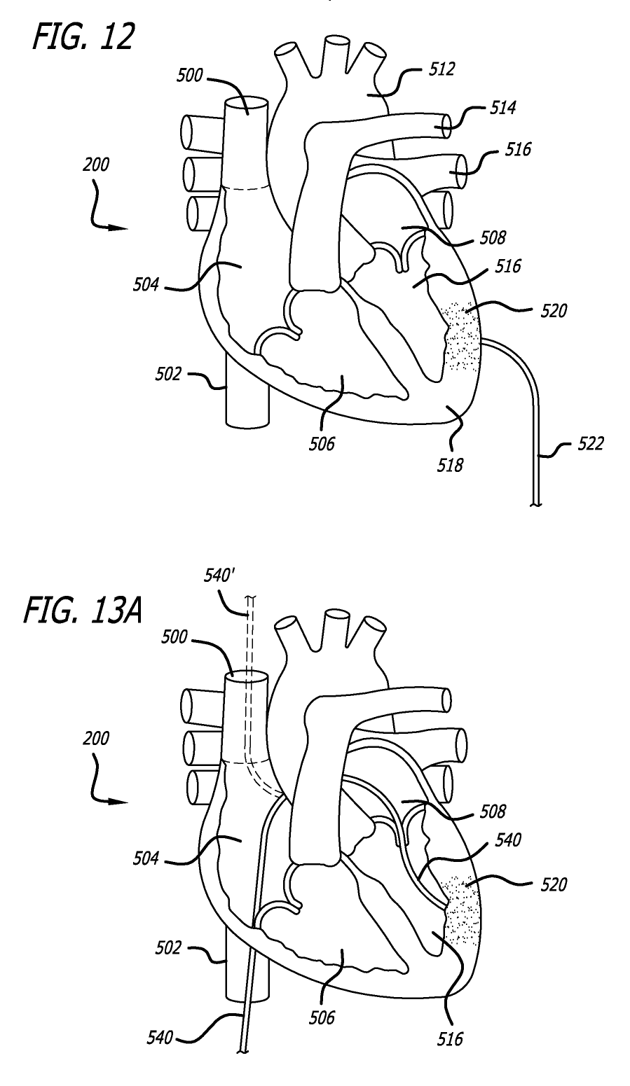

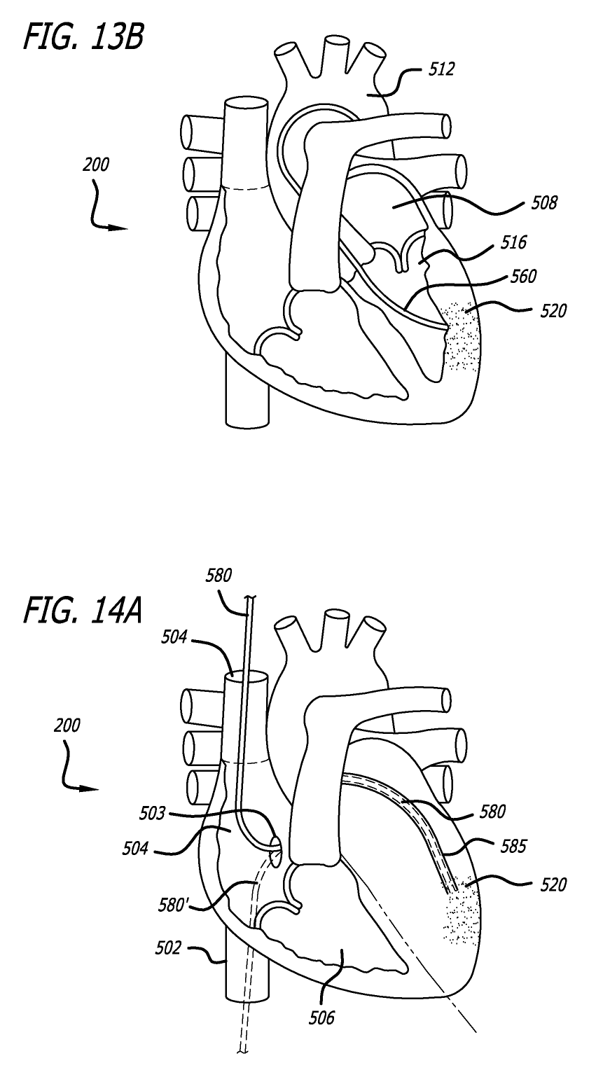

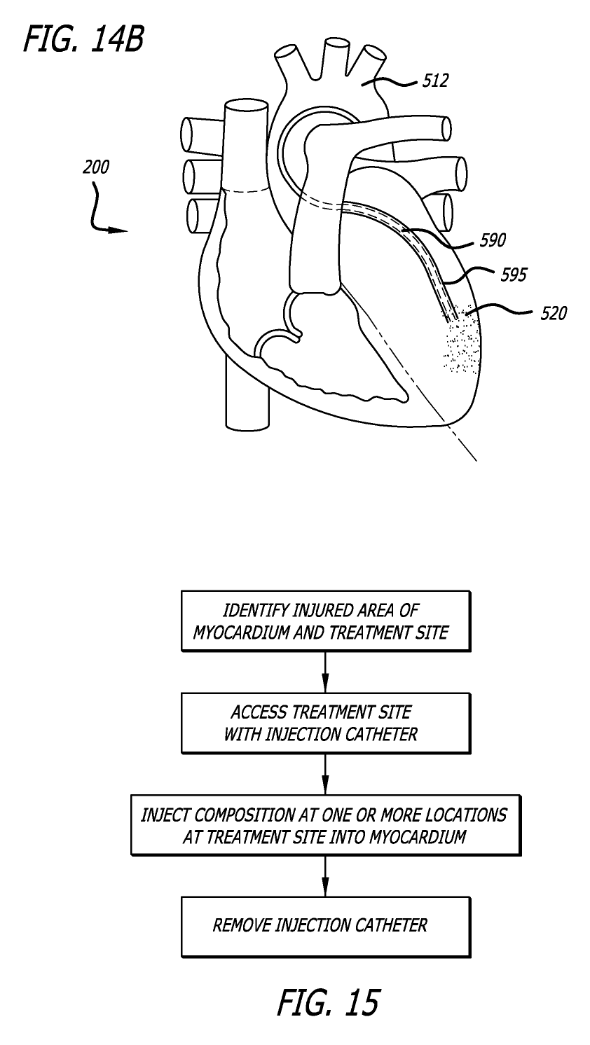

Platelet compositions of the current invention can be delivered to the cardiac wall by a catheter system. Catheter delivery systems suitable for the current invention include systems having multiple biaxial or coaxial lumens with staggered or flush tips. The catheter systems of the current invention can include needles or other injection devices located at the distal end, and syringes at the proximal end of the catheters. The catheters and other delivery devices of the current invention can have differently sized lumens to ensure that multi-component compositions can be delivered to the cardiac tissue in the desired ratio. Another embodiment of a catheter system may be used to create a composition reservoir within the cardiac wall itself to provide sustained delivery. A catheter may be introduced endovascularly into a blood vessel until the distal portion is adjacent the desired treatment location. The needle assembly may be oriented and deployed to puncture the wall of the vessel and enter the cardiac tissue. The composition can then be injected into the cardiac tissue and, thereby, form a reservoir. When catheter systems are used, a clinician can navigate to a patient's heart using one of the plurality of routes known for accessing the heart through the vasculature, or navigation to a heart chamber for delivery of the compositions epicardially (FIG. 12), endocardially (FIG. 13A-B) or transvascularly (FIG. 14A-B). In FIGS. 12 and 13 the entire heart is shown in cross section. In FIG. 14 the right ventricle and atrium are shown in cross-section while the left ventricle and atrium are shown closed with the epicardial surface and its coronary vessels in view

Epicardial delivery of platelet compositions comprises accessing a treatment site 520, in a non-limiting example, in the left ventricle 516 of a heart 200 from the epicardial, that is, exterior, surface of the heart as depicted in FIG. 12 and injecting the composition into treatment site 520 with a delivery device 522.

Endocardial delivery of platelet compositions (FIGS. 13A and B) comprises accessing a treatment site 520, for example, in the left ventricle of a heart 200, with a delivery device 540, 540′ percutaneously through an anterograde approach (FIG. 13A) through the superior vena cava 500 (delivery device 540′) or inferior vena cava 502 (delivery device 540) into the right ventricle 504. The delivery device 540 is passed through the interatrial septum into the left atrium 508 and then into the left ventricle 516 to reach treatment site 520 where the composition is injected with delivery device 540. An alternative endocardial delivery method depicted in FIG. 13B comprises accessing a treatment site 520, for example, in the left ventricle of a heart 200, with a delivery device 560 percutaneously through a retrograde approach through the aorta 512 into the left atrium 508 and then into the left ventricle 516 to reach treatment site 520 where the composition is injected with delivery device 560.

Transvascular delivery of platelet compositions (FIGS. 14A and B) comprises accessing a treatment site 520, for example, in the left ventricle of a heart 200, with delivery device 580, 580′ percutaneously through a venous approach (FIG. 14A) through the superior vena cava 500 (delivery device 580) or inferior vena cava 502 (delivery device 580′) into the right ventricle 504. The delivery device 580 is passed through the coronary sinus 503 into the cardiac venous system via these veins and, if needed, leaving these veins by tracking through cardiac tissue, it reaches treatment site 520 where the composition is injected with delivery device 580. An alternative transvascular delivery method depicted in FIG. 14B comprises accessing a treatment site 520, for example, in the left ventricle of a heart 200, with a delivery device 590 percutaneously through an arterial approach through the aorta 512 into a coronary artery 595 to reach treatment site 520 where the composition is injected with delivery device 590.

Devices for injecting the platelet compositions of the current invention can include refrigerated parts for keeping the various components of the compositions cool. Various embodiments of delivery devices for practicing the current invention can include a refrigerated/cooled chamber for thrombin refill, a refrigerated/cooled chamber for thrombin, and/or an agitator mechanism in a PRP refill or injection chamber to prevent settling of the PRP. Delivery devices can include heating or cooling devices used to heat or cool the cardiac tissue or compositions to speed up or slow down the gelling/hardening time after delivery. Some devices of the present invention can include catheters or other delivery devices with a cooled lumen or lumens for keeping components of the injected compositions cool while they are traveling through a device lumen. As noted above, some devices can include a mixing chamber for mixing the components of an injected composition before the substance is delivered into the tissue. In one embodiment of the invention, the PRP is stored in an agitating/vibrating chamber that provides sufficient agitation to keep the PRP homogeneous. In another embodiment, the clinician provides sufficient agitation to the delivery device by tilting, or otherwise manipulating the device to keep the PRP homogeneous.

A clinician practicing the current invention may need to make multiple injections using a single delivery assembly. Thus, at least one embodiment of the delivery devices of the current invention includes a device having at least one reusable needle. Some embodiments of the present invention may include delivery devices having an automated dosing system, e.g., a syringe advancing system. The automated dosing system may allow each dose to be pre-determined and dialed in (can be variable or fixed), e.g., a screw-type setting system. One embodiment of the current invention may include a proximal handle wherein each time the proximal handle is pushed; a pre-determined dose is delivered at a pre-determined or manually-controllable rate.

In further alternative embodiments, the delivery system may include a plurality of needle assemblies (similar to the individual needle assemblies described above), to be deployed in a predetermined arrangement along the periphery of a catheter. In one embodiment, the needle assemblies may be arranged in one or more rows. In particular, it may desirable to access an extended remote tissue region, for example extending substantially parallel to a vessel, within the myocardium. With a multiple needle transvascular catheter system, a single device may be delivered into a vessel and oriented. The array of needles may be sequentially or simultaneously deployed to inject a composition into the extended tissue region, thereby providing a selected trajectory pattern. Catheter based devices such as those described above are disclosed in U.S. Patent No. 6,283,951, the disclosure of which is incorporated herein by reference thereto.

If a clinician is practicing the current invention using a minimally invasive or percutaneous technique, he/she may need some sort of real-time visualization or navigation to ensure site-specific injections. Thus, at least one embodiment of the present invention uses MNav technologies to superimpose pre-operative MRI or CT images onto fluoroscopic images of a delivery catheter to track it in real-time to target sites. In one embodiment, the clinician uses a contrast agent and/or navigation technologies to track the needle-tip during injection in a virtual 3-D environment. This technique marks previous injections to ensure proper spacing of future injections.

The needle assembly (or other device component) may include a feedback element or sensor for measuring a physiological condition to guide delivery of compositions to the desired location. For example, an EKG lead may be included on the distal tip or otherwise delivered within the selected tissue region to detect and guide injection towards electrically silent or quiet areas of cardiac tissue, or to allow electrical events within the heart to be monitored during delivery of the composition. During treatment, for example, the composition may be delivered into a tissue region until a desired condition is met. Also, local EKG monitoring can be used to target and guide injection towards electrically silent or quiet areas of cardiac tissue.

Regardless of the device used to deliver the platelet composition or how the clinician accesses the cardiac wall, a clinician practicing the current invention may have the need for precise local placement and depth-control for each injection. In one embodiment of the present invention, the substance is delivered/injected to a depth in the cardiac wall that is approximately midway between the outside wall and the inside wall. In other embodiments, the substances are delivered to a depth that is closer to either the inside wall or the outside wall. The substances may be delivered intra-myocardially, sub-endocardially, or sub-epicardially. In another embodiment of the invention, the depth of the injection will vary based on the thickness of the target tissue and the depth is less at the apex of a heart than it is at other locations on the heart.

To achieve depth control, the delivery device of at least one embodiment of the present invention includes a stopper fixed (or adjustably fixed) on the needle shaft, at a desired distance from needle's distal tip, to prevent penetration into tissue beyond a specified depth. Some embodiments use the method of injecting one or more needles into tissue at a tangent to the tissue surface to control the depth of the injection. In at least one embodiment of the present invention, the needle can be positioned to inject at an angle perpendicular (90 degrees) to the tissue, tangential (0 degrees) to the tissue, or any desired angle in between. Suction can facilitate controlled positioning and entry of the injector.

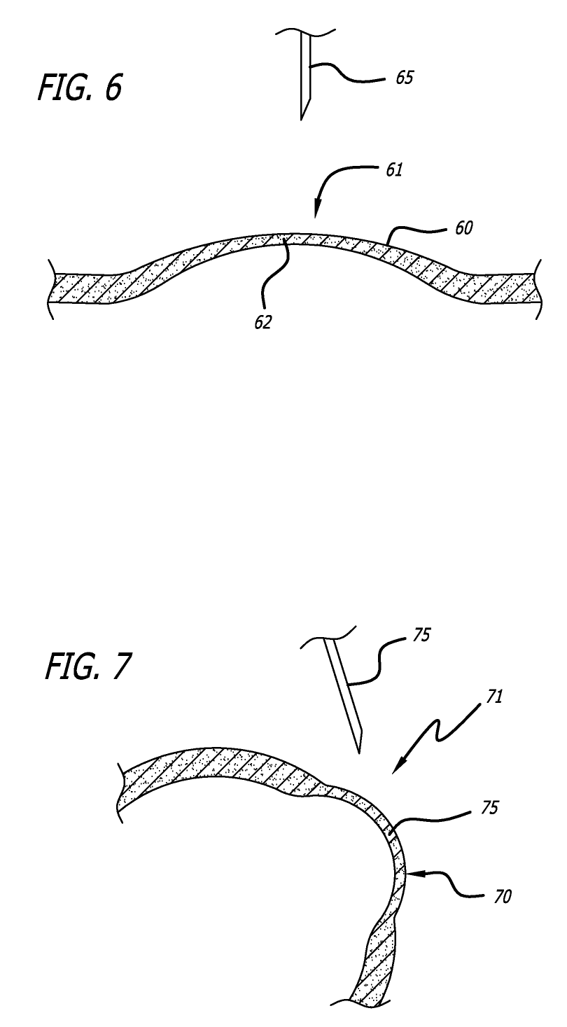

Referring now to FIG. 6, there can be seen an example of an injection according to one embodiment of the current invention wherein the needle 65 of delivery device (not shown) is approaching at an angle generally perpendicular to a remodeled portion of cardiac wall 60. The needle will puncture the cardiac wall at a point 61 directly above the desired delivery location 62 within the cardiac tissue. The device may include one or more means, for example, as described above, to ensure that the needle achieves the desired penetration depth into the myocardium.

FIG. 7 shows an example of an injection according to one embodiment of the current invention wherein the needle 75 of the delivery device (not shown) is approaching at an angle approximately tangentially to the desired injection point 71 in the cardiac wall. The needle will puncture the cardiac wall at a point 71 located a desired distance tangentially from the desired delivery location 72 within the cardiac tissue. Although not shown in the figure, a suction type stabilizer device, may be applied to the surface of the heart, at a location around or near the injection site, to stabilize the target region or the adjacent beating heart, respectively. The device will secure a generally dome shaped section of myocardium 70 therein so that the composition can be delivered. The device can include one or more means as described above to ensure that the needle achieves, but does not exceed, the desired penetration into the myocardium.

At least one embodiment of the present invention uses a “Smart-Needle” to detect distance from the needle tip to the ventricular blood compartment or endocardial surface, so that the needle tip is maintained in the cardiac wall. Such a needle can rely on imaging around or ahead of the needle tip by imaging modes such as ultrasound.

At times it might be desirable to distribute the platelet composition as widely as possible around the injection site. It might also be desirable to have the platelet composition be uniformly distributed around the injection site. One method for enhancing distribution of a platelet composition around an injection site is to use needles having holes in the side vs. using needles having holes in the end. Multiple side holes can provide a wider distribution of composition around the injection site. Side holes also provide access to the tissue from a multitude of places rather than just from the end of the needle, thereby requiring less travel of the composition for wider distribution. A potential benefit of side holes in the needles is that if the needle tip accidentally penetrates through the heart wall and into a cardiac chamber, the composition may still be injected into cardiac tissue as opposed to being injected into the blood stream within the cardiac chamber. Another method for enhancing distribution of a composition around an injection site is to increase the number of needles used at the injection site. If desired, the multi-needle delivery device of the present invention, allows for multiple needles to be placed close to each other in order to provide a uniform distribution over a larger area as compared to the use of a single needle device. The combination of side holes on the needles of a multi-needle device may provide a broad distribution of composition around an injection site.

In one embodiment of the present invention, suction may be used to improve the distribution of a composition around the injection site. The use of suction can create a negative pressure in the interstitial space. This negative pressure within the interstitial space can help the composition to travel farther and more freely, since the composition is driven by a negative pressure gradient. The combination of suction and side holes on the needles of a multi-needle device may provide a more thorough and broad distribution of composition around an injection site.

In one embodiment of the present invention, the delivery of platelet compositions from the delivery device into tissue may be enhanced via the application of an electric current, for example via iontophoresis. In general, the delivery of ionized agents into tissue may be enhanced via a small current applied across two electrodes. Positive ions may be introduced into the tissue from the positive pole, or negative ions from the negative pole. The use of iontophoresis may markedly facilitate the transport of certain ionized agents through tissue.

In one embodiment, one or more needles of the delivery device may act as the positive and/or negative poles. For example, a grounding electrode may be used in combination with a needle electrode via a monopolar arrangement to deliver an ionized composition iontophoretically to the target tissue. In one embodiment, a composition may be first dispersed from the needle into tissue. Following delivery, the composition may be iontophoretically driven deeper into the tissue via the application of an electric current. In one embodiment, a delivery device having multiple needles may comprise both the positive and negative poles via a bipolar arrangement. Further, in one embodiment, multiple needle electrodes may be used simultaneously or sequentially to inject a substance and/or deliver an electric current.

When practicing the current invention, one goal is to inject a substance into the cardiac wall while avoiding accidental delivery into one or more chambers of the heart, the coronary artery or venous system. Delivery into one or more of these areas may have negative consequences such as pulmonary or systemic embolization, stroke, cardiac congestion, and/or distant thromboembolism, for example. The current invention addresses and attempts to prevent these negative consequences in a variety of ways. In at least one embodiment of the present invention, the ratio of the components of the composition is selected so that the composition gels or polymerizes almost immediately in-situ to minimize migration of one or more of the components. In one embodiment, a balloon catheter is placed in the coronary sinus and inflated during delivery until gelling is complete. This would prevent liquid components from traveling from the tissue to the coronary venous tree and instead promote residence and gelling in the target tissue. At least one embodiment includes a pressure control system on the delivery device, to ensure that injectate pressure never exceeds ventricular chamber pressure. This would encourage retention in tissue and prevent pressure-driven migration of the composition through the thebesian venous system into the cardiac chamber. One embodiment of the present invention uses a “Smart Needle” as described above to prevent negative consequences from occurring.

At least one embodiment of the present invention includes a proximally-hand-operated distal sleeve that covers the needle tip or applies local negative pressure to prevent outward flow of component(s) from the tip of the needle between injections where multiple injections are required. In at least one embodiment, the column of components in a catheter is held under a constant minimum pressure that prevents outflow in between injections. In at least one embodiment, one-way valves may be placed within each line to prevent entry of one component into a line containing another. This is especially important when the gelling reaction is rapid and the different components need to be maintained separately until the time and site of injection. This will prevent clogging of the delivery device, which will allow repeated injections using a single device.

At least one embodiment of the present invention prevents backbleed out of the needle track, during and after removal of the needle, by keeping the needle in place for several seconds (e.g. 5-30 sec beyond the expected clotting time) following injection, to utilize the injectate as a ‘plug’ preventing back-bleed, before removing needle. In at least one embodiment of the current invention, the needle is left in place for the expected gelling time of the injected substance and then withdrawn. In one embodiment of the invention, the gelling time of an injected composition is five seconds.

Several embodiments of the current invention can include sensors and other means to assist in directing the delivery device to a desired location, ensuring that the injections occur at a desired depth, ensuring the delivery device is at the treatment site, ensuring that the desired volume of composition is delivered, and other functions that may require some type of sensor or imaging means to be used. For example, real-time recording of electrical activity (e.g., EKG), pH, oxygenation, metabolites such as lactic acid, CO₂, or other local indicators of cardiac tissue viability or activity can be used to help guide the injections to the desired location. In some embodiments of the present invention, the delivery device may include one or more sensors. For example, the sensors may be one or more electrical sensors, fiber optic sensors, chemical sensors, imaging sensors, structural sensors and/or proximity sensors that measure conductance. In one embodiment, the sensors may be tissue depth sensors for determining the depth of tissue adjacent the delivery device. In one embodiment, a sensor that detects pH, oxygenation, a blood metabolite, a tissue metabolite, etc may be used at the end of the delivery device to alert the user if and when the tip has entered the chamber blood. This would cause the operator to re-position the delivery instrument before delivering the composition. The one or more depth sensors may be used to control the depth of needle penetration into the tissue. In this way, the needle penetration depth can be controlled, for example, according to the thickness of tissue, e.g., tissue of a heart chamber wall. In some embodiments, sensors may be positioned or located on one or more needles of the delivery device. In some embodiments, sensors may be positioned or located on one or more tissue-contacting surfaces of the delivery device. In other embodiments of the present invention, the delivery device may include one or more indicators. For example, a variety of indicators, e.g., visual or audible, may be used to indicate to the physician that the desired tissue depth has been achieved.

Furthermore, the delivery device may comprise sensors to allow the surgeon or clinician to ensure the delivery device is within the heart wall rather than in the ventricle at the time of injection. Non-limiting examples of sensors which would allow determination of the location of the injector include, pressure sensors, pH sensors and sensors for dissolved gases, such as oxygen. An additional sensor that may be associated with the delivery devices suitable for use with the present invention include sensors which indicate flow of blood such as a backflow port or a backflow lumen which would inform a surgeon or clinician that the needle portion of the delivery device is in an area which has blood flow rather than within a tissue.

While the volume of platelet composition injected may vary based on the size of the heart and the area to be treated, in at least one embodiment of the present invention, about 1100 μL of platelet composition is injected into the cardiac wall per injection site. In another embodiment, about 200 μL to 1000 μL of the platelet composition is delivered per injection site. In at least one other embodiment, about 100 μL and 10000 μL of the platelet composition is delivered per injection site. In another embodiment, about 50 μL of platelet composition is injected into the cardiac wall per injection site. In one embodiment, the clinician adjusts the injection volume, the number and spacing of injection sites, and the total volume of composition per heart to optimize clinical benefit while minimizing clinical risk.

The total injection volume per heart may be dose-dependent based on the size of the heart, the size of the injured region of cardiac wall, the desired extent of structural reinforcement of the tissue, and/or the size of the area requiring revascularization. In at least one embodiment, the total volume of platelet composition injected into the cardiac wall is as much as can be accommodated by the tissue in a reasonable number of injection sites. In another embodiment, the total volume of composition injected is less than 15000 μL (15 mL).

The number of injection sites per heart can be based on the size and shape of the injured region, the desired location of the injections, and the distance separating the injection sites. In at least one embodiment, the number of injection sites can range from 5-25 sites. The distance separating injection sites will vary based on the desired volume of platelet composition to be injected per injection site, the desired total volume to be injected, and the condition of the injured tissue. In at least one embodiment, the distance between injection sites is approximately 2 cm and in at least one other embodiment, the distance between injection sites is 1 cm. In still another embodiment, the separation distance between injection sites can range between about 50 mm and about 2 cm. In another embodiment, the distance between injection sites can be in the range of 0.5 cm to 2.5 cm. In another embodiment, the distance between injection sites is greater than 2.5 cm. Injections can be continuous or interrupted along a needle track instead of as discrete single injections.

In one embodiment of the present invention, the platelet composition is injected into the cardiac tissue in a pattern that encourages formation of blood vessels. One exemplary pattern is a linear pattern that connects two target areas of tissue so that formation of blood vessels is stimulated along the linear pattern. In another embodiment, the pattern is branched. In particular, the formation of blood vessels comprises the formation of large-bore conduit vessels.

FIG. 11 schematically depicts an area of injured cardiac tissue after multiple injections of a platelet composition of the present invention. The composition is injected into the injured myocardium approximately midway between the epicardial surface 404 and the endocardial surface 406 along the plane 402 of the ventricle or chamber. The composition is injected into multiple injection sites 410, 420, 430, 440 and 450 resulting in the diffusion of injectate several centimeters from the injection site. The injected composition diffuses such that, if multiple injections are approximately 2 cm apart, the composition forms an overlapping field of structural support material. For example, composition 412 is injected at injection site 410 and diffuses as depicted in FIG. 11. Further, composition 422 is injected at injection site 420 and diffuses and intermingles with composition 412. This is repeated at injection sites 430, 440 and 450 such that compositions 412, 422, 432, 442 and 452 form a continuous overlapping field of structural support material. In this embodiment, compositions 412, 422, 432, 442, and 452 are the same composition, in a non-limited example autologous platelet gel. In another embodiment, more than one composition can be injected into a treatment site.

The location of the delivery can vary based on the size and shape of the injured region of cardiac tissue, and the desired extent of structural reinforcement of the tissue. In at least one embodiment of the present invention, the composition is delivered only into the injured cardiac tissue, while in other embodiments the peri-injury zone around the injured region is treated, and, in at least one other embodiment, the composition is delivered into only the healthy tissue that borders an injured region. In other embodiments, the composition may be delivered to any combination of the regions of injured cardiac tissue, tissue in the peri-injury zone, and healthy tissue.

The timing of platelet composition delivery relative to an injurious event will be based on the severity of the injury, the extent of the injury, the condition of the patient, and the progression of any tissue remodeling. In at least one embodiment, the platelet composition is delivered one to eight hours following an injurious event such as an MI, for example within one to eight hours following ischemia-reperfusion (in the catheterization lab setting immediately after re-perfusion). In another embodiment, the platelet composition is delivered to the cardiac wall within one hour of an injurious event. In another embodiment the platelet composition is injected three to four days after an injury (after clinical stabilization of the patient, which would make it safe for the patient to undergo a separate procedure). In at least one embodiment, the platelet composition is delivered more than one week after the injury, including up to months or years after injury. Other times for injecting compositions into the cardiac wall are also contemplated, including prior to any injurious event, and immediately upon finding an area of injured cardiac tissue (for preventing additional remodeling in older injuries). In another embodiment of the invention, compositions can be injected into the cardiac tissue years after an injurious event. In another embodiment, the platelet composition is injected into the cardiac tissue from about 1 hour to about 2 years after an injurious event. In another embodiment, the platelet composition is injected into the cardiac tissue from about 6 hours to about 1 year after an injurious event. In another embodiment, the platelet composition is injected into the cardiac tissue from about 12 hours to about 9 months after an injurious event. In another embodiment, the platelet composition is injected into the cardiac tissue from about 24 hours to about 6 months after an injurious event. In another embodiment, the platelet composition is injected into the cardiac tissue from about 48 hours to about 3 months after an injurious event. In another embodiment, the platelet composition is injected into the cardiac tissue up to about 10 years after an injurious event.

In addition to the foregoing uses for the platelet compositions, methods and systems of the present invention, it will be apparent to those skilled in the art that other injured tissues, in addition to injured cardiac tissue, would benefit from the delivery of a treatment that promotes neovascularization. Examples of such tissues include ischemic tissues in organs or sites including, but not limited to, wounds, gastrointestinal tissue, kidney, liver, skin, and neural tissue such as brain, spinal cord and nerves.

### EXAMPLES

Experiments have been conducted in laboratory conditions testing the methods and devices of the present invention disclosed herein. These include in vitro studies (described in Examples 1 and 2) in vivo studies conducted in healthy porcine tissue (Examples 3 and 4) and in vivo studies conducted in injured ovine tissue (Example 5).

### Example No. 1

Various combinations of the components for autologous platelet gel (APG) were tested in vitro using human blood, porcine blood, and ovine blood. One composition involved the extraction of 6 mL of platelet rich plasma (PRP) from 60 mL of whole blood (52.5 mL whole blood+7.5 mL anticoagulent [ACD-A, Anticoagulant Citrate Dextrose Solution A, comprising citric acid, sodium citrate and dextrose]). This PRP was combined approximately 10:1 (vol:vol) with bovine thrombin (1000 U/mL stock in 10% CaCl₂), such that mixing occured only in the targeted tissue. This was the composition tested in vivo as described below.

### Example No. 2

The ability of fibrinogen to affect the gelling and/or physical properties of APG was directly tested in vitro. PRP and platelet poor plasma (PPP) were prepared from fresh sheep blood using the Medtronic Magellan® Platelet Separator. Autologous fibrinogen was further extracted from the resulting PPP using an ethanol precipitation method. Alternative methods such as cryoprecipitation can be used for isolation of fibrinogen. The precipitated fibrinogen was re-suspended in PRP to generate autologous fibrinogen-fortified PRP (AFFPRP). Two preparations of APG were compared from the same animal—(1) conventional APG made from PRP+1000 U/ml bovine thrombin in a 10:1 ratio and (2) fibrinogen-fortified APG made from AFFPRP +1000 U/ml bovine thrombin in a 10:1 ratio. The fibrinogen-fortified APG was noticeably firmer/harder than the conventional APG generated from the same animal's blood. This confirms the utility of fibrinogen to augment the mechanical properties of APG without reducing the gelling rate.

### Example No. 3

It has been successfully demonstrated that intramural delivery of APG as two separate components (autologous PRP and bovine thrombin) that meet and clot in the tissue can be safely achieved in vivo.

Model & Access: A healthy pig model was used to test the safety and efficacy of delivery. One hundred and eighty milliliters of unheparinized blood was obtained and used to make 18 cc of PRP using a Medtronic Magellan® Autologous Platelet Separator on the day of the procedure. The animal was then heparinized to an activated clotting time (ACT) in the 250-300 range. A median sternotomy provided access to the epicardial surface of the heart.

Injections: Three injection systems were tested: System 1, a 27 gauge syringe to deliver PRP alone; System 2, an 18 gauge stainless steel needle containing a 2-lumen beveled catheter (0.0085-inch internal diameter [ID] each) with luer-lock into the needle and two independent proximal syringes (12 mL and 1 mL in size). The syringes were operated using a one-handed manifold which ensured simultaneous injection of the two components at the desired ratio (in this example, approximately 11:1). This was used to inject autologous PRP and bovine thrombin; and System 3, a suction injector which combined a suction head (to be placed on the epicardial surface of the heart) with a dual-needle injector. The suction member is driven by a vacuum pump which achieves local stabilization of the beating heart. It additionally draws the cardiac wall up into the suction cup so that the needles (entering the tissue parallel to the plane of the chamber) can be delivered at a controllable depth. The needles are driven by two separate syringes, also anchored to a one-handed injection manifold as described above as depicted in FIG. 10. A 12 mL and 1 mL syringe were used to ensure delivery of the desired ratio of autologous PRP and bovine thrombin (in this example 11:1).

Multiple injections of small volume (200-400 μl/each) were performed via an epicardial surgical approach. For injections using Systems 1 and 2 above, injections were made perpendicular to the target tissue, and a “depth stop” was used to ensure injection to a desired depth. Target depth was 5 mm in the left ventricle and 3 mm in the right ventricle. The depth-stop consisted of a C-shaped member with a central hole through which the injection needle was passed. A side-screw (which narrows the lumen size of the depth-stop as it is screwed in) was used to anchor the depth-stop along the outside of the needle at the desired position along its length. As the needle is gently advanced into the target tissue by the application of a force, the needle reaches the level of the depth-stop, beyond which it could not be advanced. Thus, this system ensures a fixed depth of needle penetration into tissue and ensures intramural injection occurs when wall thickness is known or estimatable.

For all injections in this study, the Medtronic Starfish® cardiac stabilizer (available from Medtronic, Inc., Minneapolis, Minn. USA) was used to provide procedural stabilization of the beating heart.

Target Tissue: Injections were performed in the left ventricle (LV, at its base, mid-position, and apex) and right ventricle (RV, at its base, mid-position, and apex). Injections into the LV were targeted to a 5 mm depth. Injections into the RV were targeted to a 3 mm depth.

Compositions: Different injectates were tested.
1) autologous PRP alone− to determine whether clot formation occurs in absence of exogenous thrombin
2) autologous PRP+ bovine thrombin
3) Each of the above injections was performed with and without addition of toluidine blue dye to the autologous PRP. This was to test the utility and efficacy of a tracking dye for experimental purposes.
4) Saline control

Results: Hemostasis after APG injections was excellent. Specifically, multiple left ventricular injections of up to 1000 μl/each of APG (PRP:thrombin at 10:1) into healthy porcine myocardium were feasible and clinically safe. No adverse events were observed for up to 3 days of follow-up. Multiple right ventricular injections of up to 200 μl/each of APG (PRP:thrombin at 10:1) into healthy porcine myocardium were feasible and clinically safe. No adverse events were observed over a 2 hour follow-up period.

Twenty-three injections were well-tolerated without arrhythmia, hypoxemia, or any clinical compromise during or for 1 hr following the last injection. No thrombotic or thromboembolic sequellae were found post-mortem. All 23 injections were successful, and injection sites examined during necropsy.

Furthermore, APG injection into myocardium demonstrated a protective effect against arrhythmia. In this pig model, injection of 5600 μl of APG in divided left ventricle (LV) injections rendered the heart relatively resistant to fatal arrhythmia caused by an intravascular dose of potassium chloride (KCl). Instead of developing the expected fibrillation rhythm within 10-15 seconds of a standard dose of KCl, no arrhythmias were observed for >1.5 minutes. A second dose of KCl was required before any arrhythmias developed.

Platelet gel can be formed from PRP alone without the addition of exogenous thrombin. Platelet rich plasma injected into myocardium alone (without thrombin) surprisingly gels in situ. The present inventor has formulated the non-binding hypothesis that tissue thrombin may be present in sufficient quantities to trigger this gelling reaction. Therefore, PRP may be used to create APG within the tissue when injected alone into myocardium in vivo.

### Example No. 4

Platelet rich plasma can be tracked in tissue by adding toluidine blue dye to the PRP. This dye does not noticably change the gelling characteristics (rate of gelling, extent of gelling, firmness of resultant gel) of PRP upon its combination with thrombin.

The pattern of APG distribution upon injection into myocardium was evaluated in vivo. In three pigs, injections of APG labeled with toluidine blue demonstrated that each injection results in distribution of the APG in all directions within the tissue. The greatest spread is along the plane of the ventricle. APG travels radially in the plane of the ventricle up to 1.5 cm. In some injections, APG was detected more than 1.5 cm away from the injection site. It is likely that APG travels during the gelling process until enough gelling has occurred to prohibit further spread of the material within the tissue.

### Example No. 5

The acute effects of APG injection into ischemic myocardium were studied in a sheep anterior infarct model. In this model, myocardial infarction results in deleterious structural and functional changes that occur within minutes of the injury. The early hallmarks of remodeling include ventricular dilatation, wall thinning, akinesis and often dyskinesis. Over time, these changes progress as remodeling continues. It was determined that early intervention post-infarction by providing APG to the injured myocardium can stunt this remodeling process. Additionally, such APG treatment resulted in a significant increase in neovascularization of infarcted cardiac tissue above control infarcted animals not receiving APG.

The experiments indicated that injections were safe and well tolerated when made into infarct or non-infarct tissue, and that they can be performed safely as early as 1 hr post-MI. Controlled injections were possible with or without a cardiac stabilization device, and it was possible to make the injections without exogenous cardiac pacing. Injections were made both orthogonally and obliquely to the cardiac surface at intervals of 0.5 to 2.5 cm. The total injectate volume was tested to be safe at as high as 15.0 mL per heart, and the volume of individual injections as high as 1100 μl per injection site.

In a study of 13 sheep receiving APG one hour after infarction and followed for a 2-wk follow-up period, APG reduced arrhythmia-related post-infarction mortality from the 25-30% seen in historical control animals receiving infarction alone to 8% in animals receiving infarction plus APG.

Remodeling was prevented acutely and at two weeks after infarction and injection of APG. In this study of 13 sheep, cardiac morphology and function were qualitatively assessed at different timepoints before and after APG injection. APG injection 1 hr post-MI resulted in a noticeable thickening of the ventricle wall, and a correction of post-MI dyskinesis acutely following injection. This effect was striking at 2 wks follow-up, when post-MI remodeling appeared to be partially or fully prevented versus historical control animals receiving infarction without APG injection.

In this anterior infarct model, the ventricle dilated to a diastolic volume of 152.4% of the pre-infarct volume within minutes of the infarction. The ejection fraction (EF) also dropped to 62.1% of baseline acutely after infarction. In five animals, APG was injected into the injured myocardium 1 hr after infarction. The treated hearts each received between 10 and 13.6 cc of APG in divided injections delivered into the myocardium. This treatment reduced the expected increase in post-infarct diastolic volume from 152.4% to 108.6% of the pre-infarct volume. This demonstrates a substantial effect of APG to prevent the expected post-MI increase in chamber volume, one of the key metrics of remodeling. In this study, APG injection also had a beneficial effect on post-MI EF, as it was restored from 62.1% to 70.3% of the pre-MI level. In one animal, APG delivery resulted in an EF that was 111.1% of pre-MI levels. That is, in this animal, EF was 45% at baseline, 35% immediately post-infarction, and 50% following administration of APG. This demonstrates that APG administration following cardiac injury can partially or fully reverse detrimental acute effects of infarction on EF, and in some situations may augment EF to above pre-infarct levels.

TABLE 1

| | Immediately Post-MI | Post-APG Injection |
|---|---|---|
| | (% baseline) | (% baseline) |
| Diastolic Volume | 152.4 | 108.6 |
| Ejection Fraction | 62.1 | 70.3 |

In twelve sheep receiving APG one hour after infarction and followed for 8 weeks, APG was surprisingly associated with neovascularization in the target ischemic tissue. This effect was not expected because the target tissue is, by definition, ischemic, and provides a poor environment for cells to survive, let alone grow to generate functional structures. In twelve of twelve animals, many small vessels were observed within the APG-treated infarct region at 8 weeks (FIGS. 16 and 17). Red blood cells observed within the blood vessels are highly suggestive of perfusion, implicating that these are functional vessels capable of providing fresh blood supply to surrounding tissue. Such vessels are not usually observed in animals experiencing infarction without APG injection.

The experiments revealed that there is animal-to-animal (and presumably patient-to-patient) variability in clotting rate of APG, and (to a lesser degree) the mechanical properties of APG. Methods that demonstrate improved APG clotting rate/strength include using high-dose bovine thrombin at 1000 U/mL to make APG, and using cooled (˜0° C.) thrombin to make APG. Additionally the clotting rate/strength can be improved by fortifying autologous PRP with concentrated fibrinogen (e.g., autologous fibrinogen prepared by ethanol extraction or frozen preparation). Also, the post injection clotting rate/strength can be improved by extremely careful handling of PRP prior to injection to ensure minimal pre-activation.

Several methods were identified to enhance retention of the injectate in the target tissue and to address possible leakage/backbleed issues. These methods include using high-dose bovine thrombin at 1000 U/mL to make APG. An agitator mechanism can be used in the PRP delivery and/or refill chamber to prevent settling or dissolution of the PRP. This will ensure delivery of a homogeneous PRP to the target tissue and facilitate improved clotting. Other methods include allowing the needle to dwell for 5-10 second in the injection site after the injectate has been delivered, using an oblique angle to lengthen the injection track in the tissue, and local stabilization of the injection site on entry of the needle (to prevent tearing). Each of these methods was tested in the aforementioned Examples.

Using cooled (˜0° C.) thrombin to make APG also enhances retention of the injectate in the target tissue. For the embodiment using cooled thrombin, a refrigerated/cooled chamber can be used in the thrombin delivery and/or refill chamber.

The injected compositions can be visualized by intra-operative ECHO (echocardiography), which can be used to confirm adequate needle placement and retention. The ECHO can be used as a separate device or can be included within the delivery system (e.g. similar to intravascular ultrasound [IVUS]).

Unintended perforation of a heart chamber and/or delivery into chamber blood (or blood vessels), can be avoided by using imaging guidance during injections, such as that provided by ECHO or IVUS. Additionally, it was found that direct epicardial injections into the apex of the heart should be avoided to prevent chamber puncture. Instead, oblique injections should be used to access apical tissue. Also, a device can be used to inform the operator when the delivery portion of the delivery device is in an undesired position for delivery, such as in the ventricle or in a coronary vessel. Such a device may have at least one sensor include, but not limited to, a pressure sensor, a color detector, an oxygen sensor, a carbon dioxide sensor or a lumen to express backflowing blood under pressure that generates a unique signal when the delivery system is positioned such that its target is in a blood space. Once alerted, the user can re-position the device before delivering the composition.

These experiments have shown that the methods disclosed herein can be used to restore infarct left ventricular wall thickness to (or beyond) pre-MI levels immediately following injections. This favorable effect persists (reproducibly) out to 1 week. The methods can also restore left ventricular ejection fraction (EF) to pre-MI levels immediately following injections. Additionally, treatments disclosed herein can improve cardiac dynamics and function post-MI by giving dyskinetic segments of left ventricular tissue akinetic properties.

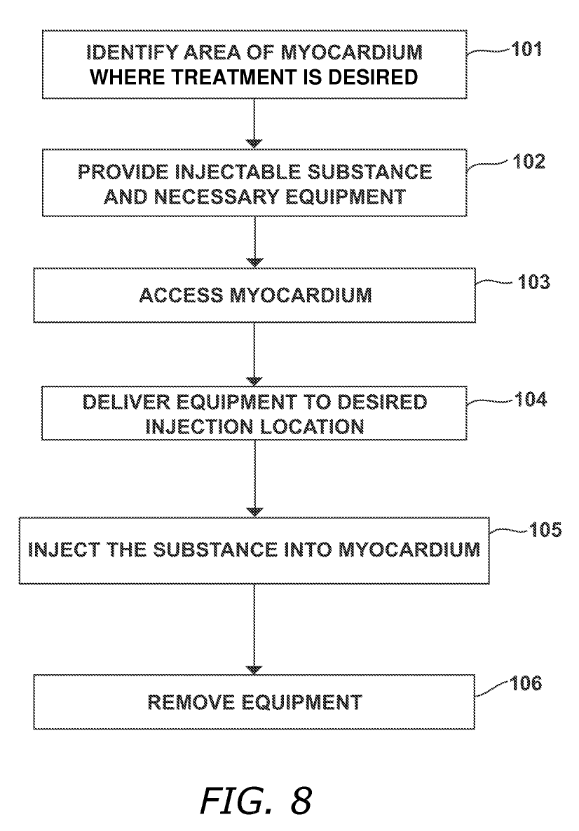

The current invention discloses a method of treating injured cardiac tissue by injecting substances that promote neovascularization with or without components that structurally reinforce the tissue. Referring to FIG. 8, the method generally comprises the steps of identifying and/or imaging the ischemic region of cardiac tissue where therapy is desired 101, determining an appropriate substance (biologic therapy with or without structural support) for injecting into the cardiac tissue to achieve the desired effect (neovascularization with or without structural reinforcement of the tissue), selecting the appropriate device for injecting the substance into the cardiac tissue 102, accessing the cardiac tissue 103, delivering the substance and delivery device to the desired treatment location 104, injecting the substance into the cardiac tissue 105 and withdrawing the device 106. The method and devices for injecting the composition (substance/injectate), the composition, and the processes for delivery have been discussed herein.

Furthermore, as seen in FIG. 15, the system of the current invention comprises identification of the injured area of cardiac tissue and the treatment site, accessing the treatment site with a delivery device, injecting the composition at one or more locations at the treatment site in the cardiac tissue and removing the delivery device from the patient.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A system for neovascularization of cardiac tissue comprising:
a platelet composition and
at least one delivery device for introducing said platelet composition into said cardiac tissue;
wherein said platelet composition induces neovascularization in said cardiac tissue.

2. The system of claim 1 wherein said platelet composition is selected from the group consisting of platelet gel, platelet rich plasma and platelet poor plasma.

3. The system of claim 1 wherein said platelet composition is autologous.

4. The system of claim 2 wherein said platelet gel is formed from platelet poor plasma or platelet rich plasma and an activating agent.

5. The system of claim 4 wherein said activating agent is thrombin.

6. The system of claim 5 wherein said thrombin is selected from the group consisting of recombinant thrombin, human thrombin, animal thrombin, engineered thrombin and autologous thrombin.

7. The system of claim 4 wherein said platelet gel is formed from platelet rich plasma or platelet poor plasma and thrombin at a ratio of between about 5:1 and about 25:1.

8. The system of claim 7 wherein said ratio of platelet rich plasma or platelet poor plasma to thrombin is about 10:1.

9. The system of claim 2 wherein said platelet composition comprises platelet rich plasma without an exogenous source of thrombin.

10. The system of claim 1 wherein said platelet composition is delivered to said treatment site and forms a gel within said cardiac tissue at said treatment site.

11. The system of claim 1 wherein said system further comprises a structural material selected from the group consisting of collagen, biocompatible polymers, alginates, synthetic/natural compounds, fibrinogen, silk-elastin polymers, hydrogels, and dental composite material.

12. The system of claim 11 wherein said structural material forms a solid or a gel as a result of physical or chemical cross-linking or activation, wherein said activation is selected from the group consisting of enzymatic, chemical, thermal or light activation of said composition.

13. The system of claim 1 wherein said platelet composition further comprises a bioactive agent.

14. The system of claim 13 wherein said bioactive agent is selected from the group consisting of pharmaceutically active compounds, hormones, growth factors, enzymes, DNA, RNA, siRNA, viruses, proteins, lipids, polymers, hyaluronic acid, antibodies, antibiotics, anti-inflammatory agents, anti-sense nucleotides and transforming nucleic acids, and combinations thereof.

15. The system of claim 1 wherein said platelet composition further comprises a contrast agent.

16. The system of claim 1 wherein said platelet composition is provided to said injured cardiac tissue between about 1 hour and about 1 year after injury occurs to said cardiac tissue.

17. The system of claim 1 wherein said platelet composition is provided in about 1 to 20 injections.

18. The system of claim 17 wherein said injections are provided sequentially.

19. The system of claim 17 wherein said injections are provided approximately simultaneously.

20. The system of claim 17 wherein said platelet composition comprises a total injection volume up to about 15 mL.

21. The system of claim 17 wherein said platelet composition comprises an injection volume up to about 1100 microliters per injection.

22. The system of claim 1 wherein said platelet composition is injected into said cardiac tissue at an angle orthogonal or oblique to the tissue surface.

23. The system of claim 1 wherein said injection site in said cardiac tissue is selected from the group consisting of sub-endocardial, sub-epicardial and intra-myocardial sites.

24. The system of claim 23 wherein said platelet composition is injected into said cardiac tissue at a depth approximately midway through the thickness of the myocardium.

25. The system of claim 1 wherein said delivery device is an injection catheter selected from the group consisting of an endocardial injection catheter, a transvascular injection catheter and an epicardial injection catheter.

26. The system of claim 1 wherein said platelet composition is provided to said treatment site during an injurious event or after an injurious event has occurred.

27. The system of claim 1 wherein said treatment site is selected from the group consisting of the injured area, the peri-injury area and the healthy tissue surrounding the injured area.

28. A method of inducing neovascularization in cardiac tissue comprising:
providing a platelet composition at a treatment site in said cardiac tissue wherein said platelet composition induces neovascularization in said cardiac tissue.

29. The method according to claim 28 wherein said platelet composition is selected from the group consisting of platelet gel, platelet rich plasma and platelet poor plasma.

30. The method according to claim 28 wherein said platelet composition is autologous.
